(19) United States
(12) Patent Application Publication
(10) Pub. No.: US 2006/0178411 A1
(43) Pub. Date: Aug. 10, 2006

(54) Sulfonimide containing compounds and their use in polymer electrolyte membranes for electrochemical cells

(76) Inventor: Mark F. Teasley, Landenberg, PA (US)

Correspondence Address: E I du Pont de Nemours & Company, Legal Patents, Wilmington, DE, 19898, US

(21) Appl. No.: 10/560,883

(22) PCT Filed: Jun. 25, 2004
(86) PCT No.: PCT/US04/20705

§ 371 Date: Dec. 13, 2005

Related U.S. Application Data
(60) Provisional application No. 60/482,925, filed on Jun. 27, 2003.

(51) Int. Cl. A61K 31/41; C07D 417/14; C07D 413/14; C07D 403/14

(52) U.S. Cl. 514/364; 548/136; 548/143; 548/156; 548/217; 548/304.7; 548/266.2

(57) ABSTRACT

A compound having the general structure (I), wherein A [symbol] 1? is a monovalent, divalent, or trivalent aromatic heterocyclic group comprising heterocyclic rings; R [symbol] 1?, R [symbol] 2?, and R [symbol] 3? are divalent fluorinated groups; m, n, and p are 0 to 3, with the proviso that m+n+p is equal to 1, 2, or 3 so that the carbon atoms of the heterocyclic rings are fully substituted by acidic fluorinated sulfonyl-containing groups; q is 0 or 1; Y [symbol] 1? is —OH, —NH—SO?2#191-R [symbol] 4? wherein R [symbol] 4? is a monovalent fluorinated group, —NH—, —NH—SO?2#191-R [symbol] 5?-SO?2#191-NH—, or —NH—SO?2#191-R [symbol] 6?-A [symbol] 2?-R [symbol] 7?-SO?2#191-NH—, wherein A [symbol] 2? is a divalent heterocyclic group and R [symbol] 5?, R [symbol] 6?, and R [symbol] 7? are divalent fluorinated groups; and Y [symbol] 2? and Y [symbol] 3? are —OH or —NH—SO?2#191-R [symbol] 4?; with the proviso that when m and n are each equal to 1, p is 0 to 1, and q is 0, Y [symbol] 1? is selected from the group consisting of —NH—, —NH—SO?2#191-R [symbol] 5?-SO?2#191-NH—, and —NH—SO?2#191-R [symbol] 6?-A [symbol] 2?-R [symbol] 7?-SO?2#191-NH—. By compound is meant either a small molecule or a repeat unit of a polymer. The invention also provides a solid polymer electrolyte membrane, a membrane electrode assembly, a gas diffusion electrode, an electrocatalyst coating composition, and a fuel cell.

### FIELD OF THE INVENTION

The present invention relates to a novel compound and its use in electrochemical cells as an electrolyte, and more particularly to the use of the compound in polymer electrolyte membranes for electrochemical cells, such as fuel cells.

### BACKGROUND OF THE INVENTION

Electrochemical cells, such as fuel cells and lithium-ion batteries, are known. Depending on the operating conditions, each type of cell places a particular set of requirements upon the electrolytes used in them. For fuel cells, this is typically dictated by the type of fuel, such as hydrogen or methanol, used to power the cell and the composition of the membrane used to separate the electrodes. Polymer electrolyte membrane fuel cells, powered by hydrogen as the fuel, could be run at higher operating temperatures than currently employed to take advantage of lower purity feed streams, improved electrode kinetics, and better heat transfer from the fuel cell stack to improve its cooling. Waste heat could also be employed in a useful fashion. However, if current fuel cells are to be operated at greater than 100° C. then they must be pressurized to maintain adequate hydration of typical polymer electrolyte membranes, such DuPont Nafion® perfluorosulfonic acid membrane, to support useful levels of proton conductivity.

There is an ongoing need to discover novel electrolytes that improve the performance of the latest generation of electrochemical cells, such as fuel cells and lithium-ion batteries. For high-temperature fuel cells, this can be addressed by developing new polymer electrolyte membrane materials that maintain adequate proton conductivity at lower levels of hydration.

### SUMMARY OF THE INVENTION

In a first aspect, the invention provides a compound having the general structure: 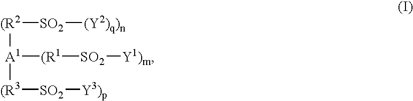
wherein A¹ is a monovalent, divalent, or trivalent aromatic heterocyclic group comprising heterocyclic rings;
R¹, R², and R³ are divalent fluorinated groups;
m, n, and p are 0 to 3, with the proviso that m+n+p is equal to 1, 2, or 3 so that the carbon atoms of the heterocyclic rings are fully substituted by acidic fluorinated sulfonyl-containing groups;
q is 0 or 1;
Y¹ is —OH, —NH—SO₂—R⁴ wherein R⁴ is a monovalent fluorinated group, —NH—, —NH—SO₂—R⁵—SO₂—NH—, or —NH—SO₂—R⁶-A²-R⁷—SO₂—NH—, wherein A² is a divalent heterocyclic group and R⁵, R⁶, and R⁷ are divalent fluorinated groups; and
Y² and Y³ are —OH or —NH—SO₂—R⁴; with the proviso that when m and n are each equal to 1, p is 0 to 1, and q is 0, Y¹ is selected from the group consisting of —NH—, —NH—SO₂—R⁵—SO₂—NH—, and —NH—SO₂—R⁶-A²-R⁷—SO₂—NH—.

By compound is meant either a small molecule or a repeat unit of a polymer. By aromatic heterocyclic group is meant any stable aromatic ring structure containing heterocyclic rings with 1, 2, or 3 carbon atoms in the heterocyclic rings available for substitution by the acidic fluorinated sulfonyl-containing groups. By fluorinated group is meant any linear, branched, or cyclic perfluorinated or partially fluorinated saturated or unsaturated group having 1 to 20 carbon atoms optionally containing ethereal oxygen, chlorine, bromine, or iodine atoms.

In the first aspect, the invention provides a compound wherein A¹ is a divalent aromatic heterocyclic group, m is 2, n and p is each equal to 0, and Y¹ is —NH—SO₂R⁴.

In the first aspect, the invention provides a compound wherein A¹ is a divalent aromatic heterocyclic group, m and n are each equal to 1, p is 0, q is 0, and Y¹ is —NH—.

In the first aspect, the invention provides a compound wherein A¹ is a divalent aromatic heterocyclic group, m and n are each equal to 1, p is 0, q is 0, and Y¹ is —NH—SO₂—R⁵—SO₂—NH—.

In the first aspect, the invention provides a compound wherein A¹ is a divalent aromatic heterocyclic group, m and n are each equal to 1, p is 0, q is 0, and Y¹ is —NH—SO₂—R⁶-A²-R⁷—SO₂—NH—.

In the first aspect, the invention provides a compound comprising a random combination of any variety of the compounds as defined above, wherein m and n are each equal to 1, p is 0 to 1, and q is 0, in any ratio with respect to each other.

In a second aspect, the invention provides a fluorinated fluorosulfonyl-substituted heterocycle having the general structure: 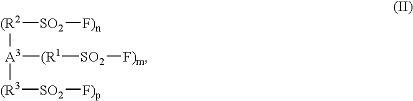
wherein A³ is a divalent or trivalent aromatic heterocyclic group comprising heterocyclic rings;
R¹, R², and R³ are divalent fluorinated groups;
m, n, and p are 0 to 3, with the proviso that m+n+p is equal to 2 or 3 so that the carbon atoms of the heterocyclic rings are fully substituted by fluorinated fluorosulfonyl groups.

In the second aspect, the invention provides a compound wherein A³ is a divalent aromatic heterocyclic group, m and n are each equal to 1, and p is 0.

In the second aspect, the invention provides a compound wherein A³ is a divalent aromatic heterocyclic group, n and p are each equal to 0, and m is 2.

In a third aspect, the invention provides a process for synthesizing a compound comprising the following steps:

(a) providing a fluorosulfonyl-containing acyl derivative having the structure:
F—SO₂R⁸—X,
wherein R⁸ is a divalent fluorinated group as defined above for R¹ and X is an acyl group selected from the group consisting of acyl fluoride, acyl chloride, acyl bromide, acyl iodide, an ester, an amide, and nitrile;

(b) condensing the fluorosulfonyl-containing acyl derivative from step (a) with a nitrogenous reagent, such as ammonia; hydrazine; azide, such as sodium azide; organic ortho-substituted aromatic amine, such as ortho-phenylene diamine; etc. to form a sulfonyl-containing precursor;

(c) cyclizing the sulfonyl-containing precursor of step (b) by thermolysis or dehydration to form a sulfonyl-containing aromatic heterocyclic compound; and

(d) converting the sulfonyl-containing aromatic heterocyclic compound of step (c), containing fluorosulfonyl groups or sulfonamide groups, into an acidic sulfonyl-containing aromatic heterocyclic compound by either:
(i) condensing fluorosulfonyl groups with a fluorinated sulfonamide,
(ii) condensing sulfonamide groups with a fluorinated sulfonyl fluoride,
(iii) condensing fluorosulfonyl groups first with ammonia to form sulfonamide groups followed by a fluorinated sulfonyl fluoride to form sulfonimide groups, or
(iv) hydrolysis of fluorosulfonyl or sulfonamide groups to form sulfonic acid groups.

In the third aspect, the invention also provides a process for synthesizing a bis(sulfonimide)[1,3,4]oxadiazole by condensing a fluorosulfonyl acyl fluoride, F—SO₂—R⁸—CO—F, with hydrazine to form a bis(fluorosulfonyl)dihydrazide containing a dihydrazide group and fluorosulfonyl groups; forming a [1,3,4]oxadiazole ring by cyclizing the dihydrazide group using dehydration; condensing the fluorosulfonyl groups with ammonia to form a bis(sulfonamide)-[1,3,4]oxadiazole containing sulfonamide groups; and forming sulfonimide groups by condensing a fluorinated sulfonyl fluoride, R⁴—SO₂—F, with the sulfonamide groups.

In the third aspect, the invention also provides a process for synthesizing a copolymer containing sulfonimide and [1,3,4]oxadiazole groups by condensing a fluorosulfonyl acyl fluoride, F—SO₂—R⁸—CO—F, with hydrazine to form a bis(fluorosulfonyl)dihydrazide containing a dihydrazide group and fluorosulfonyl groups; forming a [1,3,4]oxadiazole ring by cyclizing the dihydrazide group using dehydration; condensing the fluorosulfonyl groups with ammonia to form a bis(sulfonamide)-[1,3,4]oxadiazole containing sulfonamide groups; and forming sulfonimide groups by condensing a fluorinated disulfonyl difluoride, F—SO₂—R⁵—SO₂—F, with the sulfonamide groups.

In the third aspect, the invention also provides a process for synthesizing a benzimidazole sulfonimide by condensing a fluorosulfonyl acyl fluoride, F—SO₂—R⁸—CO—F, with ammonia to form a diamide containing a carbamide group and a sulfonamide group; condensing the carbamide group with an ortho-phenylene diamine to form a carbamide adduct; cyclizing the carbamide adduct by thermolysis to form a benzimidazole group, and forming a sulfonimide group by condensing a fluorinated sulfonyl fluoride, R⁴—SO₂—F, with the sulfonamide group.

In the third aspect, the invention also provides a process for synthesizing a benzimidazole sulfonic acid by condensing a fluorosulfonyl acyl fluoride, F—SO₂—R⁸—CO—F, with an ortho-phenylene diamine to form a carbamide adduct; cyclizing the carbamide adduct by thermolysis to form a benzimidazole group, and forming a sulfonic acid group by hydrolyzing the fluorosulfonyl group.

In a fourth aspect, the invention provides a solid polymer electrolyte membrane comprising a compound having the general structure: 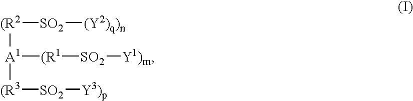
wherein A¹ is a monovalent, divalent, or trivalent aromatic heterocyclic group comprising heterocyclic rings;
R¹, R², and R³ are divalent fluorinated groups;
m, n, and p are 0 to 3, with the proviso that m+n+p is equal to 1, 2, or 3 so that the carbon atoms of the heterocyclic rings are fully substituted by acidic fluorinated sulfonyl-containing groups;
q is 0 or 1;
Y¹ is —OH, —NH—SO₂—R⁴ wherein R⁴ is a monovalent fluorinated group, —NH—, —NH—SO₂—R⁵—SO₂—NH—, or —NH—SO₂—R⁶-A²-R⁷—SO₂NH—, wherein A² is a divalent aromatic heterocyclic group and R⁵, R⁶, and R⁷ are divalent fluorinated groups; and
Y² and Y³ are —OH or —NH—SO₂—R⁴; with the proviso that when m and n are each equal to 1, p is 0 to 1, and q is 0, Y¹ is selected from the group consisting of —NH—, —NH—SO₂—R⁵—SO₂—NH—, and —NH—SO₂—R⁶-A²-R⁷—SO₂—NH—.

By polymer electrolyte is meant a compound as defined above that possesses acidic fluorinated sulfonyl-containing groups, such as sulfonic acid and sulfonimide groups, whose protons can optionally be exchanged for other cations, such as lithium and sodium, for use in electrochemical cells, such as fuel cells, lithium-ion batteries, and chloralkali cells.

In the fourth aspect, the invention provides a solid polymer electrolyte membrane further comprising a porous support.

In a fifth aspect, the invention provides a membrane electrode assembly comprising a solid polymer electrolyte membrane having a first surface and a second surface, an anode present on the first surface of the solid polymer electrolyte membrane, and a cathode present on the second surface of the solid polymer electrolyte membrane, wherein the solid polymer electrolyte membrane comprises a compound having the general structure: 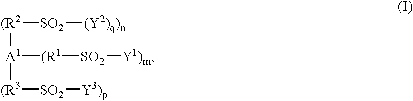
wherein A¹ is a monovalent, divalent, or trivalent aromatic heterocyclic group comprising heterocyclic rings;
R¹, R², and R³ are divalent fluorinated groups;
m, n, and p are 0 to 3, with the proviso that m+n+p is equal to 1, 2, or 3 so that the carbon atoms of the heterocyclic rings are fully substituted by acidic fluorinated sulfonyl-containing groups;
q is 0 or 1;
Y¹ is —OH, —NH—SO₂—R⁴ wherein R⁴ is a monovalent fluorinated group, —NH—, —NH—SO₂—R⁵—SO₂—NH—, or —NH—SO₂—R⁶-A²-R⁷—SO₂—NH—, wherein A² is a divalent aromatic heterocyclic group and R⁵, R⁶, and R⁷ are divalent fluorinated groups; and
Y² and Y³ are —OH or —NH—SO₂R⁴; with the proviso that when m and n are each equal to 1, p is 0 to 1, and q is 0, Y¹ is selected from the group consisting of —NH—, —NH—SO₂—R⁵—SO₂—NH—, and —NH—SO₂—R⁶-A²-R⁷—SO₂—NH—.

In the fifth aspect, the invention provides a membrane electrode assembly comprising a polymer electrolyte membrane, wherein the polymer electrolyte membrane further comprises a porous substrate.

In a sixth aspect, the invention provides an electrochemical cell, such as a fuel cell, comprising a solid polymer electrolyte membrane, wherein the solid polymer electrolyte membrane comprises a compound having the general structure: 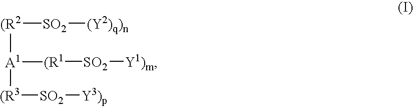
wherein A¹ is a monovalent, divalent, or trivalent aromatic heterocyclic group comprising heterocyclic rings;
R¹, R², and R³ are divalent fluorinated groups;
m, n, and p are 0 to 3, with the proviso that m+n+p is equal to 1, 2, or 3 so that the carbon atoms of the heterocyclic rings are fully substituted by acidic fluorinated sulfonyl-containing groups;
q is 0 or 1;
Y¹ is —OH, —NH—SO₂—R⁴ wherein R⁴ is a monovalent fluorinated group, —NH—, —NH—SO₂—R⁵—SO₂—NH—, or —NH—SO₂—R⁶-A²-R⁷—SO₂—NH—, wherein A² is a divalent aromatic heterocyclic group and R⁵, R⁶, and R⁷ are divalent fluorinated groups; and
Y² and Y³ are —OH or —NH—SO₂—R⁴; with the proviso that when m and n are each equal to 1, p is 0 to 1, and q is 0, Y¹ is selected from the group consisting of —NH—, —NH—SO₂—R⁵—SO₂—NH—, and —NH—SO₂—R⁶-A²-R⁷—SO₂—NH—.

In the sixth aspect, the invention provides a fuel cell comprising a polymer electrolyte membrane, wherein the polymer electrolyte membrane further comprises a porous substrate.

In the sixth aspect, the fuel cell further comprises an anode and a cathode present on the first and second surfaces of the solid polymer electrolyte membrane.

In the sixth aspect, the fuel cell further comprises a gas diffusion backing present on the side away from the anode or cathode present on the first and second surfaces of the solid polymer electrolyte membrane.

In the sixth aspect, the fuel cell comprises gas diffusion electrodes, wherein the gas diffusion electrodes comprise at least one electrode on a gas diffusion backing, wherein the electrode is present in contact with the solid polymer electrolyte membrane.

In the sixth aspect, the fuel cell further comprises a means for delivering a fuel to the anode, a means for delivering oxygen to the cathode, a means for connecting the anode and cathode to an external electrical load, a fuel in the liquid or gaseous state in contact with the anode, and oxygen in contact with the cathode. Some suitable fuels include hydrogen; alcohols such as methanol and ethanol; ethers such as diethyl ether, etc. The oxygen can be delivered to the cathode as a pure gas, in a mixture with other inert gases, or as a constituent of air.

### BRIEF DESCRIPTION OF DRAWINGS

FIG. 1 is a schematic illustration of a single cell assembly.

FIG. 2 is a schematic illustration of the lower fixture of a four-electrode cell for in-plane conductivity measurement.

### DETAILED DESCRIPTION OF THE INVENTION

This invention concerns novel compounds containing fluorinated sulfonyl-containing groups and aromatic heterocyclic groups, and their use as electrolytes in electrochemical cells, such as in fuel cells, batteries, chloralkali cells, electrolysis cells, sensors, electrochemical capacitors, and modified electrodes. Suitable fluorinated sulfonyl-containing groups are highly ionizable and acidic, and include partially or fully fluorinated sulfonimides, R—SO₂—NH—SO₂—R, or sulfonic acids, R—SO₃H, typically perfluorinated sulfonimides, R^{f}—SO₂—NH—SO₂—R^{f}. Suitable aromatic heterocyclic groups include oxadiazoles, triazoles, thiadiazoles, pyrazoles, triazines, tetrazoles, oxazoles, thiazoles, imidazoles, benzoxazoles, benzothiazoles, benzimidazoles, benzobisoxazoles, benzobisthiazoles, benzobisimidazoles, bibenzoxazoles, bibenzothiazoles, and bibenzimidazoles, typically [1,3,4]oxadiazoles, [1,3,4]thiadiazoles, and [1,2,4]triazoles, and most typically [1,3,4]oxadiazoles. Such aromatic heterocyclic groups have stable aromatic ring structures with 1, 2, or 3 carbon atoms in the heterocyclic ring available for substitution by the acidic fluorinated sulfonyl-containing groups. These groups can be incorporated together in various ways to obtain the novel compounds so that the resulting electrolytes remain acidic, typically highly acidic. This can be accomplished by maintaining an excess of acidic fluorinated sulfonyl-containing groups with respect to the aromatic heterocyclic groups. As their appropriate salt derivatives, these compounds have use as electrolytes in batteries, such lithium-ion batteries.

The compounds have the general structure: 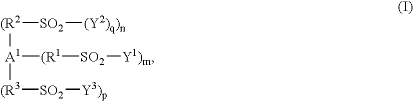
wherein A¹ is a monovalent, divalent, or trivalent aromatic heterocyclic group comprising heterocyclic rings, such as an oxadiazole, triazole, thiadiazole, pyrazole, triazine, tetrazole, oxazole, thiazole, imidazole, benzoxazole, benzothiazole, benzimidazole, benzobisoxazole, benzobisthiazole, benzobisimidazole, bibenzoxazole, bibenzothiazole, or bibenzimidazole, typically oxadiazole, triazole, thiadiazole, pyrazole, triazine, oxazole, thiazole, imidazole, benzobisoxazole, benzobisthiazole, benzobisimidazole, bibenzoxazole, bibenzothiazole, or bibenzimidazole, more typically [1,3,4]oxadiazole, [1,3,4]thiadiazole or [1,2,4]triazole, and most typically [1,3,4]oxadiazole;
R¹, R², and R³ are divalent fluorinated groups, such as any linear, branched, or cyclic perfluorinated or partially fluorinated saturated or unsaturated groups having 1 to 20 carbon atoms optionally containing ethereal oxygen, chlorine, bromine, or iodine atoms, typically linear or branched perfluorinated saturated or unsaturated groups having 1 to 10 carbon atoms optionally containing ethereal oxygen atoms, and most typically linear perfluorinated saturated groups having 1 to 6 carbon atoms;
m, n, and p are 0 to 3, with the proviso that m+n+p is equal to 1, 2, or 3, typically 2 or 3, and more typically 2, so that the carbon atoms of the heterocyclic rings are fully substituted by acidic fluorinated sulfonyl-containing groups;
q is 0 or 1;
Y¹ is —OH, —NH—SO₂—R⁴ wherein R⁴ is a monovalent fluorinated group, —NH—, —NH—SO₂—R⁵—SO₂—NH—, or —NH—SO₂—R⁶-A²-R⁷—SO₂NH—, wherein A² is a divalent aromatic heterocyclic group, such as an oxadiazole, triazole, thiadiazole, benzobisoxazole, benzobisthiazole, benzobisimidazole, bibenzoxazole, bibenzothiazole, or bibenzimidazole, typically [1,3,4]oxadiazole, [1,3,4]thiadiazole, or [1,2,4]triazole, and most typically [1,3,4]oxadiazole, and R⁵, R⁶, and R⁷ are divalent fluorinated groups as defined for R¹; and
Y² and Y³ are —OH or —NH—SO₂—R⁴; with the proviso that when m and n are each equal to 1, p is 0 to 1, and q is 0, Y¹ is selected from the group consisting of —NH—, —NH—SO₂—R⁵—SO₂—NH—, and —NH—SO₂—R⁶-A²-R⁷—SO₂—NH—.

For a small molecule, Y¹, Y², and Y³ are each equal to —OH or —NH—SO₂—R⁴, wherein R⁴ is any monovalent fluorinated group, such as any linear, branched, or cyclic perfluorinated or partially fluorinated saturated or unsaturated group having 1 to 20 carbon atoms optionally containing ethereal oxygen, chlorine, bromine, or iodine atoms, typically a linear or branched perfluorinated saturated or unsaturated group having 1 to 10 carbon atoms optionally containing ethereal oxygen atoms, and most typically a linear perfluorinated saturated group having 1 to 6 carbon atoms, and q is 1. Typically, m+n+p is equal to 2 or 3. More typically, Y¹ is —NH—SO₂—R⁴, n and p are each equal to 0 and m is 2 or 3, and most typically m is 2.

For sulfonimide step-growth polymers, structure (I) serves as the repeat unit for the polymers, [—I—], where A¹ is a divalent or trivalent aromatic heterocyclic group, such as an oxadiazole, triazole, thiadiazole, pyrazole, triazine, oxazole, thiazole, imidazole, benzobisoxazole, benzobisthiazole, benzobisimidazole, bibenzoxazole, bibenzothiazole, or bibenzimidazole, typically oxadiazole, triazole, thiadiazole, benzobisoxazole, benzobisthiazole, benzobisimidazole, bibenzoxazole, bibenzothiazole, or bibenzimidazole, more typically [1,3,4]oxadiazole, [1,3,4]thiadiazole, or [1,2,4]triazole, and most typically [1,3,4]oxadiazole; n and m are each equal to 1, p is 0 or 1, typically p is 0; q is 0; Y¹ is —NH—, —NH—SO₂—R⁵—SO₂—NH—, or —NH—SO₂—R⁶-A²-R⁷—SO₂—NH— wherein A² is a divalent aromatic heterocyclic group, such as an oxadiazole, triazole, thiadiazole, benzobisoxazole, benzobisthiazole, benzobisimidazole, bibenzoxazole, bibenzothiazole, or bibenzimidazole, typically [1,3,4]oxadiazole, [1,3,4]thiadiazole, or [1,2,4]triazole, and more typically [1,3,4]oxadiazole, and R^{5 , R}⁶, and R⁷ are divalent fluorinated groups as defined for R¹; and Y³ is —OH or —NH—SO₂—R⁴.

For a homopolymer, A¹ is typically a divalent aromatic heterocyclic group, such as an oxadiazole, triazole, thiadiazole, benzobisoxazole, benzobisthiazole, benzobisimidazole, bibenzoxazole, bibenzothiazole, or bibenzimidazole, more typically [1,3,4]oxadiazole, [1,3,4]thiadiazole, or [1,2,4]triazole, and most typically [1,3,4]oxadiazole; p is 0; and Y¹ is —NH—, which generates the following dependent structure for the repeat unit.

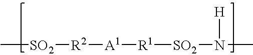

For a copolymer, A¹ is typically a divalent aromatic heterocyclic group, such as an oxadiazole, triazole, thiadiazole, benzobisoxazole, benzobisthiazole, benzobisimidazole, bibenzoxazole, bibenzothiazole, or bibenzimidazole, more typically [1,3,4]oxadiazole, [1,3,4]thiadiazole, or [1,2,4]triazole, and most typically [1,3,4]oxadiazole; p is 0; and Y¹ is —NH—SO₂R⁵—SO₂—NH—, which generates the following dependent structure for the repeat unit.

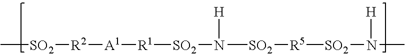

Alternatively, for another copolymer, A¹ is typically a divalent aromatic heterocyclic group, such as an oxadiazole, triazole, thiadiazole, benzobisoxazole, benzobisthiazole, benzobisimidazole, bibenzoxazole, bibenzothiazole, or bibenzimidazole, more typically [1,3,4]oxadiazole, [1,3,4]thiadiazole, or [1,2,4]triazole, and most typically [1,3,4]oxadiazole; p is 0; and Y¹ is —NH—SO₂—R⁶-A²-R⁷—SO₂NH—, which generates the following dependent structure for the repeat unit.

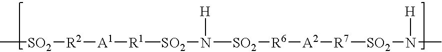

It shall be understood to one skilled in the art that a random copolymer can also be obtained by randomly combining any variety of the polymer repeat units as defined above in any ratio with respect to each other.

The synthesis of some of these novel compounds requires the use of novel fluorinated fluorosulfonyl-substituted heterocycles as key intermediates. Divalent and trivalent heterocycles substituted with fluorinated fluorosulfonyl groups are particularly useful for the preparation of the polymers either as monomers or as monomer precursors.

These fluorinated fluorosulfonyl-substituted heterocycles have the general structure: 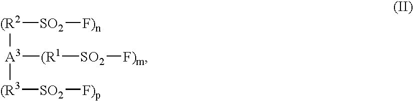
wherein A³ is a divalent or trivalent aromatic heterocyclic group, such as an oxadiazole, triazole, thiadiazole, pyrazole, triazine, oxazole, thiazole, imidazole, benzobisoxazole, benzobisthiazole, benzobisimidazole, bibenzoxazole, bibenzothiazole, or bibenzimidazole, typically oxadiazole, triazole, thiadiazole, benzobisoxazole, benzobisthiazole, benzobisimidazole, bibenzoxazole, bibenzothiazole, or bibenzimidazole, more typically [1,3,4]oxadiazole, [1,3,4]thiadiazole, or [1,2,4]triazole, and most typically [1,3,4]oxadiazole;
R¹, R², and R³ are divalent fluorinated groups, such as any linear, branched, or cyclic perfluorinated or partially fluorinated saturated or unsaturated groups having 1 to 20 carbon atoms optionally containing ethereal oxygen, chlorine, bromine, or iodine atoms, typically linear or branched perfluorinated saturated or unsaturated groups having 1 to 10 carbon atoms optionally containing ethereal oxygen atoms, and most typically linear perfluorinated saturated groups having 1 to 6 carbon atoms; and
m, n, and p are 0 to 3, with the proviso that m+n+p is equal to 2 or 3 so that the carbon atoms of the heterocyclic rings are fully substituted by fluorinated fluorosulfonyl groups, typically m and n are each equal to 1 and p is 0, and most typically n and p are each equal to 0 and m is 2.

These novel compounds and intermediates can be synthesized in various ways starting with suitable acyl derivatives represented by the general structure FSO₂—R⁸—X, wherein R⁸ is a divalent fluorinated group as defined for R¹, and X is an acyl fluoride, acyl chloride, acyl bromide, acyl iodide, an ester, an amide, or nitrile, and more typically acyl fluoride. The acyl derivatives can be condensed with suitable nitrogenous reagents, such as ammonia, hydrazine, azides, such as sodium azide, or organic ortho-substituted aromatic amines, such as ortho-phenylene diamine, to generate aromatic heterocyclic rings or their precursors, which can be cyclized to give their aromatic heterocyclic rings. The schemes below show several embodiments for the general preparation of typical compounds and polymers.

In one embodiment shown in Scheme 1, a fluorosulfonyl acyl fluoride is condensed with hydrazine to give a dihydrazide. The dihydrazide is then condensed with a fluorinated sulfonamide followed by cyclization using oleum to give a bis(sulfonimide)oxadiazole.

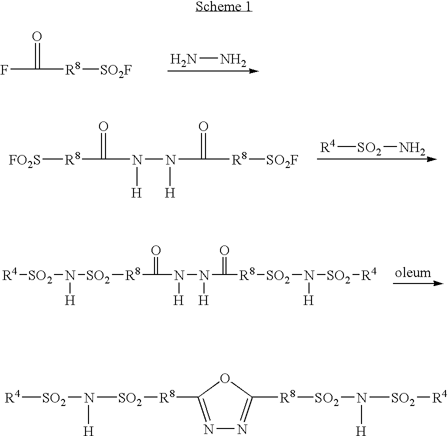

In a second embodiment shown in Scheme 2, a dihydrazide is cyclized directly to a bis(fluorosulfonyl)oxadiazole using oleum. The bis(fluorosulfonyl)oxadiazole is then condensed with ammonia to give a bis(sulfonamide)oxadiazole followed by a fluorinated sulfonyl fluoride to give a bis(sulfonimide)oxadiazole.

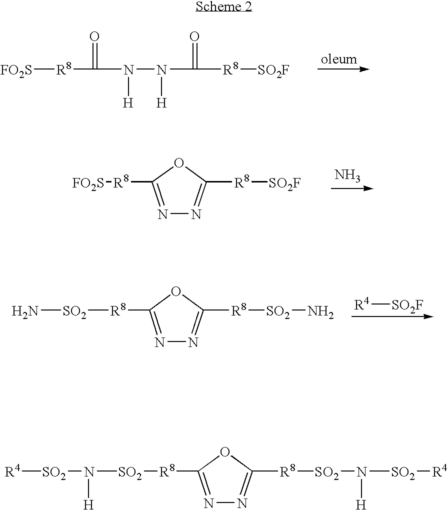

In a third embodiment shown in Scheme 3, a bis(sulfonamide)oxadiazole is reacted with a fluorinated disulfonyl difluoride to give a copolymer.

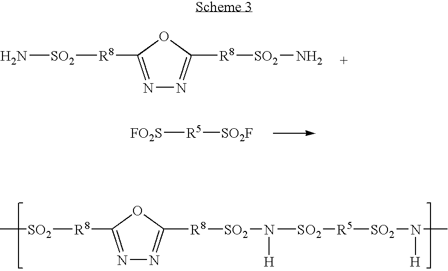

In a fourth embodiment shown in Scheme 4, a fluorosulfonyl acyl fluoride is condensed with ammonia to give a diamide followed by ortho-phenylene diamine to give a benzimidazole sulfonamide. The benzimidazole sulfonamide is condensed with a fluorinated sulfonyl fluoride to give a benzimidazole sulfonimide.

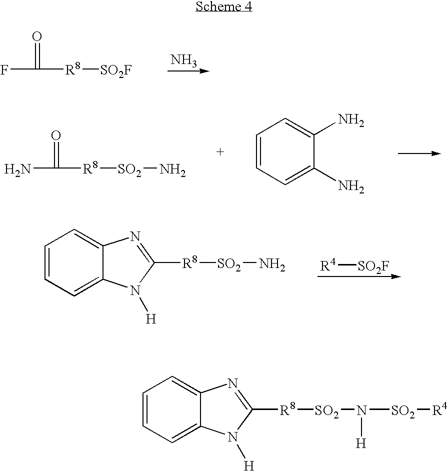

In a fifth embodiment shown in Scheme 5, a fluorosulfonyl acyl fluoride is condensed directly with ortho-phenylene diamine and hydrolyzed to give a benzimidazole sulfonic acid. 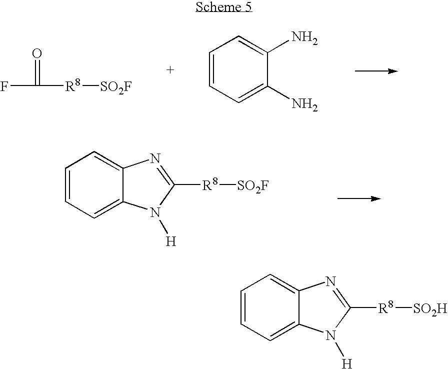
Polymer Electrolyte Membrane:

The compounds identified above are imbibed into a porous support to form a solid polymer electrolyte membrane. Typically these membranes are capable of operating at a temperature of at least 100° C. and a relative humidity of less than 50% while maintaining a proton conductivity of at least 50 mS/cm, more typically of at least 20 mS/cm at a temperature of at least 120° C. and a relative humidity of less than 25%.

Porous Support:

The porous support of the polymer electrolyte membrane may be made from a wide range of components. The porous support of the present invention may be made from a fluorinated polymer such as polychlorotrifluoroethylene, polytetrafluoroethylene, polyethylene-co-tetrafluoroethylene, and polyvinylidene difluoride, a ceramic such as silica, alumina, and silicon carbide, or a glass such as borosilicate glass. For resistance to thermal and chemical degradation, the porous polymer support typically is made of a highly fluorinated polymer, most typically a perfluorinated polymer.

For example, the polymer for the porous support can be a microporous film of polytetrafluoroethylene (PTFE) or a copolymer of tetrafluoroethylene with other perfluoroalkyl olefins or with perfluorovinyl ethers. Microporous PTFE films and sheeting are known which are suitable for use as a support layer. For example, U.S. Pat. No. 3,664,915 discloses uniaxially stretched film having at least 40% voids. U.S. Pat. Nos. 3,953,566, 3,962,153 and 4,187,390 disclose porous PTFE films having at least 70% voids.

Alternatively, the porous support may be made from fibers of the support materials discussed above by forming the fibers into a paper or a fabric woven using various weaves such as the plain weave, basket weave, leno weave, or others. A membrane suitable for the practice of the invention can be made by coating the porous support paper or fabric with the compound of the invention to form a composite membrane. To be effective the coating must be on both the outside surfaces as well as distributed through the internal pores of the support. This may be accomplished by impregnating the porous support with a solution or dispersion of the polymer suitable for the practice of the invention using a solvent that is not harmful to the polymer or the support, and under impregnation conditions that can form a thin, even coating of the polymer on the support. The support with the solution/dispersion is dried to form the membrane. If desired, thin films of a polymer electrolyte can be laminated to one or both sides of the impregnated porous support to prevent bulk flow through the membrane that can occur if large pores remain in the membrane after impregnation.

It is preferred for the compound to be present as a continuous phase within the solid polymer electrolyte membrane.

Other forms of the solid polymer electrolyte membrane include the PTFE yarn embedded type and the PTFE fibril dispersed type, wherein the PTFE fibril is dispersed in the polymer electrolyte as disclosed in 2000 Fuel Cell Seminar (10/30 to 11/2, 2000, Portland, Oreg.) Abstracts, page 23.

The compound imbibed into the porous support to form the solid polymer electrolyte membrane may be modified to contain reactive functional groups, either as side chains or end groups, that can be used to provide crosslinking, grafting, or chain extension of the compound to insure that the compound stays within the porous support and cannot be leached out when in use in a fuel cell. Some suitable reactive functional groups include vinyl, trifluorovinyl ether, trifluorovinyl phenyl ether, trifluorovinylbenzene (α,β,β-trifluorostyrene), pentafluorophenyl ether, perfluorinated nitrile, perfluorinated bromides, perfluorinated iodides, and alkoxysilanes. Alternatively, the modified or unmodified compound can be mixed with reagents and processed as needed to provide crosslinking, grafting, or chain extension.

Electrochemical Cell:

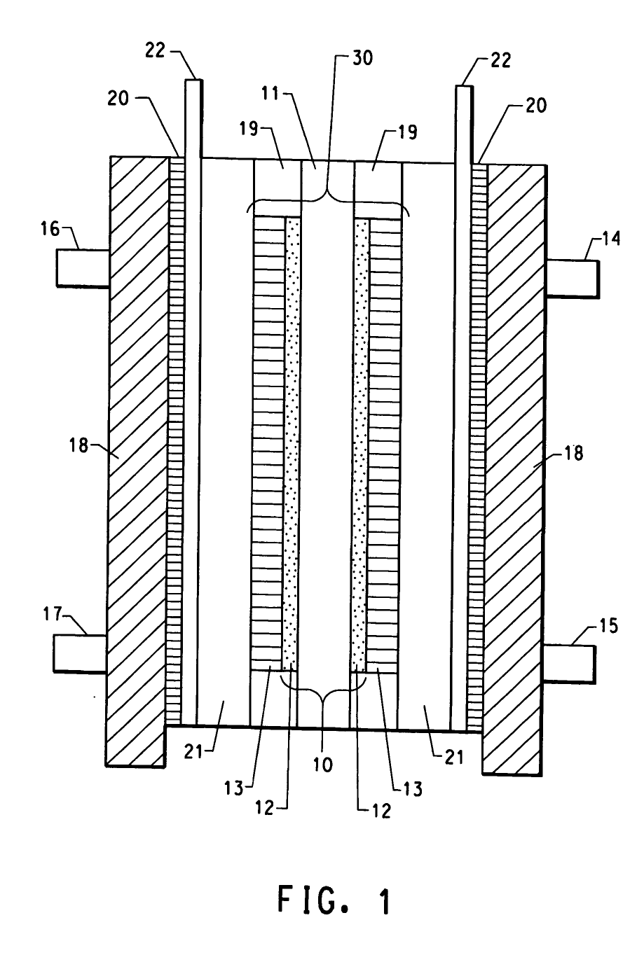

As shown in FIG. 1, an electrochemical cell, such as a fuel cell comprises a catalyst-coated membrane (CCM) (10) in combination with at least one gas diffusion backing (GDB) (13) to form an unconsolidated membrane electrode assembly (MEA). The catalyst-coated membrane (10) comprises an polymer electrolyte membrane (11) discussed above and catalyst layers or electrodes (12) formed from an electrocatalyst coating composition. The fuel cell is further provided with an inlet (14) for fuel, such as hydrogen; liquid or gaseous alcohols, e.g. methanol and ethanol; or ethers, e.g. diethyl ether, etc., an anode outlet (15), a cathode gas inlet (16), a cathode gas outlet (17), aluminum end blocks (18) tied together with tie rods (not shown), a gasket for sealing (19), an electrically insulating layer (20), graphite current collector blocks with flow fields for gas distribution (21), and gold plated current collectors (22).

Alternately, gas diffusion electrodes comprising a gas diffusion backing having a layer of an electrocatalyst coating composition thereon may be brought into contact with a solid polymer electrolyte membrane to form the MEA.

The electrocatalyst coating compositions used to apply the catalyst layers as electrodes on the CCM (10) or the GDE comprises a combination of catalysts and binders dispersed in suitable solvents for the binders, and may include other materials to improve electrical conductivity, adhesion, and durability. The catalysts may be unsupported or supported, typically on carbon, and may differ in composition depending on their use as anodes or cathodes. The binders typically consist of the same polymer used to form the polymer electrolyte membrane (11), but may contain in part or be solely composed of other suitable polymer electrolytes as needed to improve the operation of the fuel cell. The compounds of this invention may be used either singly or together in the binders or polymer electrolyte membrane (11) with other suitable polymer electrolytes, such as Naflon® perfluorosulfonic acid and sulfonated polyether sulfones, or their membranes.

The fuel cell utilizes a fuel source that may be in the gas or liquid phase, and may comprise hydrogen, an alcohol, or an ether. The fuel is humidified to the degree required to maintain adequate ionic conductivity in the solid polymer electrolyte membrane discussed above so that the fuel cell provides a high power output. Depending on the operating temperature, the fuel cell may be operated at elevated pressures to maintain the required degree of humidification. Typically a gaseous humidified hydrogen feed or methanol/water solution is supplied to the anode compartment, and air or oxygen supplied to the cathode compartment.

Catalyst Coated Membrane:

A variety of techniques are known for CCM manufacture, which apply an electrocatalyst coating composition similar to that described above onto a solid polymer electrolyte membrane. Some known methods include spraying, painting, patch coating and screen, decal, pad or flexographic printing.

In one embodiment of the invention, the MEA (30), shown in FIG. 1, may be prepared by thermally consolidating the gas diffusion backing (GDB) with a CCM at a temperature of under 200° C., preferably 140-160° C. The CCM may be made of any type known in the art. In this embodiment, an MEA comprises a solid polymer electrolyte (SPE) membrane with a thin catalyst-binder layer disposed thereon. The catalyst may be supported (typically on carbon) or unsupported. In one method of preparation, a catalyst film is prepared as a decal by spreading the electrocatalyst coating composition on a flat release substrate such as Kapton® polyimide film (available from the DuPont Company). After the coating dries, the decal is transferred to the surface of the SPE membrane by the application of pressure and heat, followed by removal of the release substrate to form a catalyst coated membrane (CCM) with a catalyst layer having a controlled thickness and catalyst distribution. Alternatively, the catalyst layer is applied directly to the membrane, such as by printing, and then the catalyst film is dried at a temperature not greater than 200° C.

The CCM, thus formed, is then combined with a GDB to form the MEA (30). The MEA is formed, by layering the CCM and the GDB, followed by consolidating the entire structure in a single step by heating to a temperature no greater than 200° C., preferably in the range of 140-160° C., and applying pressure. Both sides of the MEA can be formed in the same manner and simultaneously. Also, the composition of the catalyst layer and GDB could be different on opposite sides of the membrane.

The invention is illustrated in the following examples.

### EXAMPLES

Liquid Conductivity Measurement

The conductivity of a liquid was measured using a cell capable of handling corrosive samples at elevated temperature with volumes as small as 800 μL. Two coil electrodes were formed 25 mm apart by wrapping 0.38 mm diameter platinum wires five times around one end of a 5.14 mm diameter Macor® machinable glass ceramic rod (Corning Inc., Corning, N.Y.) and insulating the remainder of the wire leads with heat-shrink PTFE tubing. The sample was loaded into a 9 mm outside diameter×6.8 mm inside diameter×178 mm length glass tube and the rod was inserted so that the electrodes were completely immersed in the sample. The tube was placed into a forced-convection thermostated oven for heating. The real part of the AC impedance, Rₛ, was measured at a frequency of 1 kHz using a potentiostat/frequency response analyzer (PC4/750™ with EIS software, Gamry Instruments, Warminster, Pa.). The phase angles were typically less than 2 degrees, which indicates that the measurement was unaffected by capacitive contributions from the electrode interfaces. The cell constant, K, was determined by measuring the real part of the impedance, R_{c}, at a frequency of 10 kHz using a NIST traceable potassium chloride conductivity calibration standard for nominal 0.1 S/cm (0.1027 S/cm actual) and calculating as
K=R_{c}×0.1027 S/cm×(1+ΔT×0.02° C.⁻¹)
where ΔT was the difference between the temperature of the calibration standard, Tₘ, and 25° C. (ΔT=Tₘ−25). The cell constant was typically close to 12 cm⁻¹. The conductivity, κ, of the sample was then calculated as
κ=K/Rₛ
Through-Plane Conductivity Measurement

The through-plane conductivity of a membrane was measured by a technique in which the current flowed perpendicular to the plane of the membrane. The lower electrode was formed from a 12.7 mm diameter stainless steel rod and the upper electrode was formed from a 6.35 mm diameter stainless steel rod. The rods were cut to length, and their ends were polished and plated with gold. A stack was formed consisting of lower electrode/GDE/membrane/GDE/upper electrode. The GDE (gas diffusion electrode) was a catalyzed ELAT® (E-TEK Division, De Nora North America, Inc., Somerset, N.J.) comprising a carbon cloth with microporous layer, platinum catalyst, and 0.6-0.8 mg/cm² Nafion® application over the catalyst layer. The lower GDE was punched out as a 9.5 mm diameter disk, while the membrane and the upper GDE were punched out as 6.35 mm diameter disks to match the upper electrode. The stack was assembled and held in place within a block of Macor® machinable glass ceramic (Corning Inc., Corning, N.Y.) that had a 12.7 mm diameter hole drilled into the bottom of the block to accept the lower electrode and a concentric 6.4 mm diameter hole drilled into the top of the block to accept the upper electrode. A force of 270 N was applied to the stack by means of a clamp and calibrated spring. This produced a pressure of 8.6 MPa in the active area under the upper electrode, which insured a low impedance ionic contact between the GDE's and the membrane. The fixture was placed inside a forced-convection thermostated oven for heating. The real part of the AC impedance of the fixture containing the membrane, Rₛ, was measured at a frequency of 100 kHz using a potentiostat/frequency response analyzer (PC4/750™ with EIS software, Gamry Instruments, Warminster, Pa.). The fixture short, R_{f}, was also determined by measuring the real part of the AC impedance at 100 kHz for the fixture and stack assembled without a membrane sample. The conductivity, κ, of the membrane was then calculated as
κ=t/((Rₛ−R_{f})×0.317 cm²),
where t was the thickness of the membrane in cm.
In-Plane Conductivity Measurement

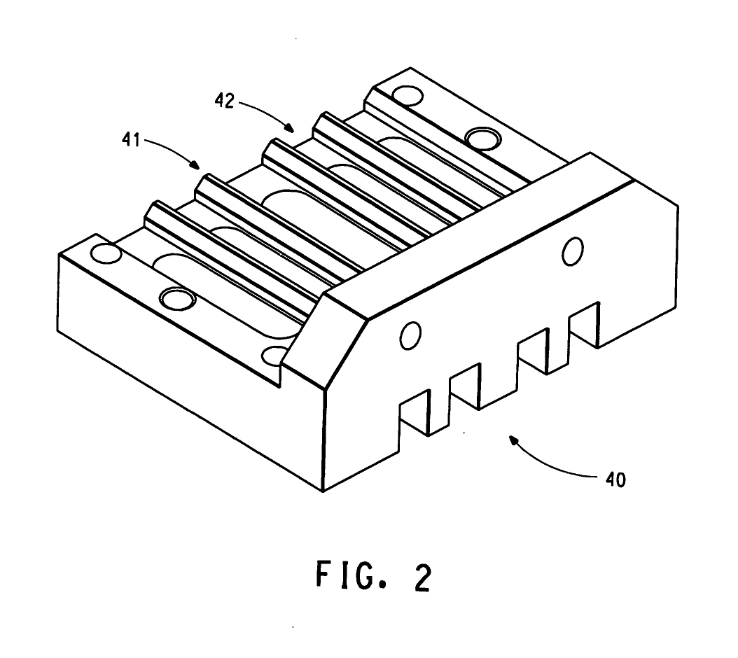

The in-plane conductivity of a membrane was measured under conditions of controlled relative humidity and temperature by a technique in which the current flowed parallel to the plane of the membrane. A four-electrode technique was used similar to that described in an article entitled “Proton Conductivity of Nafion® 117 As Measured by a Four-Electrode AC Impedance Method” by Y. Sone et al., J. Electrochem. Soc., 143, 1254 (1996), which is herein incorporated by reference. Referring to FIG. 2, a lower fixture (40) was machined from annealed glass-fiber reinforced PEEK to have four parallel ridges (41) containing grooves that supported and held four 0.25 mm diameter platinum wire electrodes. The distance between the two outer electrodes was 25 mm, while the distance between the two inner electrodes was 10 mm. A strip of membrane was cut to a width between 10 and 15 mm and a length sufficient to cover and extend slightly beyond the outer electrodes, and placed on top of the platinum electrodes. An upper fixture (not shown), which had ridges corresponding in position to those of the bottom fixture, was placed on top and the two fixtures were clamped together so as to push the membrane into contact with the platinum electrodes. The fixture containing the membrane was placed inside a small pressure vessel (pressure filter housing), which was placed inside a forced-convection thermostated oven for heating. The temperature within the vessel was measured by means of a thermocouple. Water was fed from a calibrated Waters 515 HPLC pump (Waters Corporation, Milford, Mass.) and combined with dry air fed from a calibrated mass flow controller (200 sccm maximum) to evaporate the water within a coil of 1.6 mm diameter stainless steel tubing inside the oven. The resulting humidified air was fed into the inlet of the pressure vessel. The total pressure within the vessel (100 to 345 kPa) was adjusted by means of a pressure-control let-down valve on the outlet and measured using a capacitance manometer (Model 280E, Setra Systems, Inc., Boxborough, Mass.). The relative humidity was calculated assuming ideal gas behavior using tables of the vapor pressure of liquid water as a function of temperature, the gas composition from the two flow rates, the vessel temperature, and the total pressure. Referring to FIG. 2, the slots (42) in the lower and upper parts of the fixture allowed access of humidified air to the membrane for rapid equilibration with water vapor. Current was applied between the outer two electrodes while the resultant voltage was measured between the inner two electrodes. The real part of the AC impedance (resistance) between the inner two electrodes, R, was measured at a frequency of 1 kHz using a potentiostat/frequency response analyzer (PC4/750™ with EIS software, Gamry Instruments, Warminster, Pa.). The conductivity, K, of the membrane was then calculated as
κ=1.00 cm/(R×t×w),
where t was the thickness of the membrane and w was its width (both in cm).

### Example 1

A 500 mL two-neck round bottom flask equipped with a stirring bar, gas inlet, and septum was charged with 10.5 g of poly(4-vinylpyridine), 2% cross-linked (0.1 moles), and purged with nitrogen. A solution of 18.1 g of difluoro-fluorosulfonyl-acetyl fluoride (0.1 moles) in 100 mL ether was prepared inside a glove box, added to the flask by cannula, and cooled to 0° C. A solution of 1.60 g of hydrazine (0.05 moles) in 200 mL of ether was prepared inside a glove box and slowly added to the flask by cannula. The mixture was stirred for 30 minutes, and then warmed to room temperature. The solids were filtered off and washed with ether. The filtrate was evaporated and the residue dried under vacuum. The solids were recrystallized from 100 mL of benzene using decolorizing carbon. The off-white crystals of N,N′-bis(2,2-difluoro-2-fluorosulfonyl-acetyl)hydrazine, as shown by the following structure, weighed 12.1 g for a 69% yield. ¹⁹F NMR (CD₃CN): −106.3 (d, J=5.5 Hz, CF₂), 39.7 (t, J=5.5 Hz, SO₂F). MS (CI): m/z 352.93239 (M+H, 3.6 ppm error), calculated 352.933674 (C₄H₂N₂O₆F₆S₂+H).

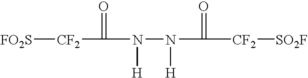

### Example 2

A 300 mL round bottom flask equipped with a stirring bar and reflux condenser was charged with 15.70 g of N,N′-bis(2,2-difluoro-2-fluorosulfonyl-acetyl)hydrazine (0.0446 moles) and 13.3 g of trifluoromethanesulfonamide (0.0892 moles) inside a glove box. The solids were dissolved in 100 mL of tetrahydrofuran and 25 mL of triethylamine (0.180 moles) was added to give a single phase. The solution was heated to a reflux overnight under nitrogen. The solvents were evaporated and the oil dried under vacuum to give N,N′-bis(N-trifluoromethylsulfonyl-2,2-difluoro-2-sulfamoyl-acetyl)hydrazine, triethylammonium salt as shown by the following structure. ¹⁹F NMR (CD₃CN): −108.9 (s, CF₂), −80.3 (s, CF₃).

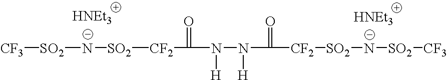

The oil was dissolved in 25 mL of 30% oleum (0.180 moles SO₃) and heated to 60° C. overnight. The solution was poured onto 200 g of ice and the flask rinsed with water. A heavy dark oil was separated from the aqueous phase, which was then extracted five times with 50 mL of dichloromethane. The combined organic phase became homogeneous when dried with magnesium sulfate, and was filtered, evaporated, and dried under vacuum. The oil weighed 32.3 g for 91% crude yield of 2,5-bis(N-trifluoromethanesulfonyl-difluoro-sulfamoyl-methyl)-[1,3,4]oxadiazole, triethylammonium salt as shown by the following structure. ¹⁹F NMR (CD₃CN): −106.8 (s, CF₂), −80.3 (s, CF₃). ¹H NMR (CD₃CN): 1.25 (t, 3H), 3.15 (m, 2H).

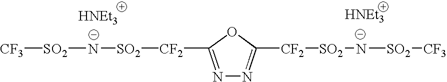

The oil was dissolved in 100 mL of tetrahydrofuran and treated with 7.5 mL of 50% sodium hydroxide to liberate the triethylamine. The solvent was evaporated and the residue dried under vacuum. The sodium salt weighed 25.4 g for a 90% crude yield and was free of triethylamine according to ¹H NMR. ¹⁹F NMR (CD₃CN): −108.6 (s, CF₂), −80.6 (s, CF₃).

The sodium salt was dissolved in hot deionized water and treated with decolorizing carbon and filter aid. The slurry was filtered and the cake washed with deionized water. The aqueous solution was concentrated to 100 mL, treated with 10 mL concentrated hydrochloric acid, and extracted twice with ether. The aqueous solution was re-treated with decolorizing carbon and filter aid, and evaporated to give a gummy residue. The residue was dissolved in 200 mL of methanol and dried with sodium sulfate. The inorganic solids were filtered off and washed with methanol. The hazy solution was re-dried and filtered through a filter aid. The clear solution was diluted with a little deionized water and evaporated to give 21.83 g. The residue was treated with 10 mL concentrated hydrochloric acid and dissolved in deionized water. The water was evaporated and the residue dried under vacuum. The residue was dissolved in acetonitrile and diluted with ether to precipitate the inorganic salts. The salts were filtered off through acid-washed filter aid. The filtrate was diluted with a little deionized water, evaporated, and dried under vacuum to give 20.2 g of an oil. ¹⁹F NMR (CD₃CN): −110.6 (s, CF₂), −80.4 (s, CF₃). Elemental analysis indicated incomplete ion exchange as the oil still contained 1.57% sodium (21 mole %).

An ion-exchange column containing 200 g of Dowex® 50WX8-1 00 was washed with methanol until the elutant was colorless followed by deionized water. The column was conditioned by eluting with 250 mL 1M hydrochloric acid followed by deionized water until the elutant was neutral. A solution of 6.1 g of the oil in 600 mL of deionized water was eluted through the column followed by 300 mL deionized water. The acidic elutant was collected to give about 900 mL of solution. The water was evaporated at 50° C. at 30 Torr to give 7 g of a light orange hydrated oil of 2,5-bis(N-trifluoromethanesulfonyl-difluoro-sulfamoyl-methyl)-[1,3,4]oxadiazole as shown by the following structure. A sample was dried under vacuum for elemental analysis, which indicated that it contained at least 23% water. Elemental analysis showed only 265 ppm sodium. ¹⁹F NMR (CD₃CN): −110.6 (s, CF₂), −80.4 (s, CF₃).

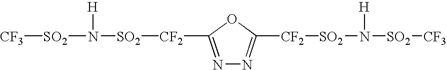

### Example 3

A glass tube was charged with 0.999 g of the hydrated oil of the bis(perfluorosulfonimide)oxadiazole from Example 2 and 0.176 g of deionized water. The liquid conductivity was measured as the tube was heated to 150° C. and cooled in 25° C. steps. The sample lost 0.257 g of water. The results are shown in Table 1.

TABLE 1

| | Temperature, ° C. | | | | | |
|---|---|---|---|---|---|---|
| | 25 | 50 | 75 | 100 | 125 | 150 |
| Conductivity (heating), | 171 | 251 | 300 | 355 | 190 | 152 |
| mS/cm | | | | | | |
| Conductivity (cooling), | 38 | 85 | 107 | 137 | 135 | — |
| mS/cm | | | | | | |

### Example 4

A glass tube was charged with 1.304 g of the hydrated oil of the 30 bis(perfluorosulfonimide)oxadiazole from Example 2 and analyzed for liquid conductivity. The tube was twice heated to 150° C. and cooled in 25° C. steps. The sample lost 0.103 g of water on the first cycle and 0.016 g on the second cycle. The results are shown in Table 2.

TABLE 2

| | Temperature, ° C. | | | | | |
|---|---|---|---|---|---|---|
| | 25 | 50 | 75 | 100 | 125 | 150 |
| Conductivity (1^{st} heat), mS/cm | 86 | 118 | 169 | 219 | 247 | 136 |
| Conductivity (1^{st} cool), mS/cm | 43 | 81 | 114 | 137 | 178 | — |
| Conductivity (2^{nd} heat), mS/cm | 35 | 54 | 82 | 120 | 148 | 158 |
| Conductivity (2^{nd} cool), mS/cm | 34 | 71 | 100 | 128 | 159 | — |

### Example 5

The ion-exchange procedure of Example 2 was repeated with 10.1 g of the impure bis(perfluorosulfonimide)oxadiazole from Example 2 dissolved in 1 L of deionized water. The resulting light orange hydrated oil weighed 13.4 g and contained at least 30% water. This oil was combined with about 4 g of the purified bis(perfluorosulfonimide)oxadiazole from Example 2 in a 100 mL round bottom flask. The hydrated oil was dried under vacuum to remove the free water. The flask was attached to a Kugelrohr bulb-to-bulb distillation apparatus and evacuated under high vacuum. The flask was heated slowly to 140° C. to liberate bound water. Once the vacuum reached 0.1 Torr, a white solid began to form in the collection bulb. The deliquescent solid weighed 7.3 g and contained a 64.5:35.5 mixture of trifluoromethanesulfonamide and the bis(perfluorosulfonimide)oxadiazole according to ¹⁹F NMR.

The ion-exchange procedure of Example 2 was repeated with the deliquescent solid dissolved in 500 mL of deionized water. The resulting light hydrated oil weighed 6.3 g and contained the pure bis(perfluoro-sulfonimide)oxadiazole. ¹⁹F NMR (CD₃CN): −110.6 (s, CF₂), −80.4 (s, CF₃).

### Example 6

This example used a modified ceramic fiber sheet (product # ASPA-1; ZIRCAR Ceramics, Inc., Florida, N.Y.). The unmodified sheet nominally contained 51% silica, 45% alumina, and 4% hydrocarbon binder. The hydrocarbon binder was burned off in an 800° C. furnace for 4 hours. The sheet was then saturated with a 2% solution of PVDF (Kynar® 741 resin; ATOFINA Chemicals, Inc., Philadelphia, Pa.) in dimethylacetamide, allowed to sit for 5 minutes, and dried in a vacuum oven at 110° C. for 1 hour. The dried modified sheet was compressed in a Carver hot press at 120° C. to give a smooth substrate containing 18% Kynar® as binder.

A 1×4 cm strip of the modified ceramic fiber substrate was cut from the stock sheet and weighed 72 mg. The strip was laid inside a vial and saturated with the hydrated oil from Example 5. The vial was heated to 80° C. in a drying oven under nitrogen to anneal the sample. The vial was cooled to room temperature and the excess oil was removed from the sample by blotting between sheets of lint-free paper. The treated strip weighed 577 mg, so it contained 87.5% of the hydrated bis(perfluorosulfonimide)oxadiazole.

The sample was 400 μm thick and 11.17 mm wide as measured for in-plane conductivity. The temperature and humidity were equilibrated at each condition to obtain the measurements shown in Table 3. The sample weighed only 287 mg after the test. The decrease in conductivity with relative humidity indicated partial dehydration of the bis(perfluorosulfonimide)oxadiazole. The high conductivity value observed at 80° C. and 95% relative humidity indicated rehydration under these conditions, which led to the weight loss.

TABLE 3

| Temperature | Relative | Resistance | Conductivity |
|---|---|---|---|
| ° C. | Humidity % | ohm | mS/cm |
| 80 | 20 | 231 | 97 |
| 120 | 23 | 169 | 132 |
| 150 | 9.5 | 2987 | 7.5 |
| 80 | 95 | 101 | 221 |

### Example 7

This example used a sample of porous Kynar® film prepared according to the procedure described in J.-M. Tarascon, et al. Solid State Ionics, 1996, 86-88, pages 49-54. An acetone dispersion was made containing 33 parts PVDF-HFP copolymer (Kynar® Powerflex® LBG resin; ATOFINA Chemicals, Inc., Philadelphia, Pa.), 22 parts fumed silica (Cabot TS530, Cabot Corporation, Boston, Mass.), and 44 parts dibutyl phthalate. The dispersion was cast to yield a film that was 50 μm thick upon drying. The dibutyl phthalate was extracted twice with excess diethyl ether.

A 1×4 cm strip of the porous film was cut from the stock sheet and weighed 19 mg. The strip was laid inside a vial and saturated with the hydrated oil from Example 5. The vial was heated to 80° C. in a drying oven under nitrogen to anneal the sample. The vial was cooled to room temperature and the excess oil was removed from the sample by blotting between sheets of lint-free paper. The treated strip weighed 46 mg, so it contained 59% of the hydrated bis(perfluorosulfonimide)oxadiazole.

The sample was 50 μm thick and 11.07 mm wide as measured for in-plane conductivity. The temperature and humidity were equilibrated at each condition before obtaining the measurements shown in Table 4.

TABLE 4

| Temperature | Relative | Resistance | Conductivity |
|---|---|---|---|
| ° C. | Humidity % | ohm | mS/cm |
| 80 | 20 | 40,816 | 4.4 |
| 120 | 23 | 16,921 | 11 |
| 80 | 95 | 608 | 297 |

### Example 8

The sample was prepared as described in Example 6, except that the strip of the modified ceramic sheet was 1×3 cm in size and weighed 51 mg. The treated strip weighed 320 mg, so it contained 84% of the hydrated bis(perfluorosulfonimide)oxadiazole.

The sample was 430 μm thick as measured for through-plane conductivity. The fixture had a short impedance of 0.240 ohms. The sample was heated to 150° C. and then cooled in 25° C. steps to obtain the measurements shown in Table 5.

TABLE 5

| | Temperature, ° C. | | | | | |
|---|---|---|---|---|---|---|
| | 150 | 125 | 100 | 75 | 50 | 25 |
| Conductivity, mS/cm | 49.3 | 49.5 | 44.0 | 37.3 | 26.6 | 12.7 |

### Example 9

The sample was prepared as described in Example 6, except that the strip of the modified ceramic sheet was 1×3 cm in size and weighed 58 mg. The treated strip weighed 359 mg, so it contained 84% of the hydrated bis(perfluorosulfonimide)oxadiazole.

The sample was 378 μm thick and 11.37 mm wide as measured for in-plane conductivity. The sample was held at 120° C. and 50% relative humidity under pressure for 40 minutes to obtain the measurements shown in Table 6. The sample weighed 216 mg after the test. The decline in conductivity and the weight loss indicated that the bis(perfluorosulfonimide)oxadiazole was partially lost from the sample under these conditions.

TABLE 6

| Time | Temperature | Relative | Pressure | Resistance | Conductivity |
|---|---|---|---|---|---|
| minute | ° C. | Humidity % | psia | ohm | mS/cm |
| 0 | 120 | 50 | 35.2 | 63 | 369 |
| 20 | 120 | 50 | 35.8 | 319 | 73 |
| 40 | 120 | 50 | 35.8 | 335 | 69 |

### Example 10

The sample was prepared as described in Example 6, except that the strip of the modified ceramic sheet was 1×3 cm in size and weighed 53 mg. The treated strip weighed 350 mg, so it contained 85% of the hydrated bis(perfluorosulfonimide)oxadiazole.

The sample was 387 μm thick and 11.55 mm wide as measured for in-plane conductivity. The temperature and humidity were equilibrated at each condition before obtaining the measurements shown in Table 7. The regain in conductivity under the final conditions indicated that the bis(perfluorosulfonimide)oxadiazole was thermally stable.

TABLE 7

| Temperature | Relative | Resistance | Conductivity |
|---|---|---|---|
| ° C. | Humidity % | ohm | mS/cm |
| 80 | 20 | 249 | 90 |
| 120 | 23 | 176 | 127 |
| 150 | 9.5 | 5139 | 4.4 |
| 80 | 95 | 97 | 231 |

### Example 11

The sample was prepared as described in Example 6, except that the strip of the modified ceramic sheet was 1×3 cm in size and weighed 54 mg. The treated strip weighed 344 mg, so it contained 84% of the hydrated bis(perfluorosulfonimide)oxadiazole.

The sample was 422 μm thick and 10.74 mm wide as measured for in-plane conductivity. The temperature and humidity were equilibrated at each condition before obtaining the measurements shown in Table 8. The sample weighed 284 mg after the test. The inability to regain higher conductivity under the final conditions and the drop in conductivity at 140-150° C. and 23% relative humidity was caused by the partial loss of the bis(perfluorosulfonimide)oxadiazole due to excessive hydration under those conditions.

TABLE 8

| Temperature | Relative | Pressure | Resistance | Conductivity |
|---|---|---|---|---|
| ° C. | Humidity % | psia | ohm | mS/cm |
| 120 | 10 | 14.8 | 397 | 56 |
| 120 | 23 | 14.9 | 223 | 99 |
| 120 | 23 | 30.0 | 237 | 93 |
| 130 | 23 | 29.7 | 220 | 100 |
| 140 | 23 | 29.9 | 366 | 60 |
| 150 | 23 | 30.0 | 1602 | 14 |
| 120 | 23 | 29.9 | 3275 | 6.7 |

### Example 12

The sample was prepared as described in Example 6, except that the strip of the modified ceramic sheet was 1×3 cm in size and weighed 54 mg. The treated strip weighed 415 mg, so it contained 87% of the hydrated bis(perfluorosulfonimide)oxadiazole.

The sample was 380 μm thick and 10.92 mm wide as measured for in-plane conductivity. The sample was held at 120° C. and 23% relative humidity for 15 hours to obtain the measurements shown in Table 9. The sample weighed 384 mg after the test, so it only lost 7.5%. The weight loss and the drop in conductivity were due to slow dehydration under these conditions.

TABLE 9

| Time | Temperature | Relative | Resistance | Conductivity |
|---|---|---|---|---|
| hour | ° C. | Humidity % | ohm | mS/cm |
| 0 | 120 | 23 | 139 | 174 |
| 15 | 120 | 23 | 577 | 42 |

### Example 13

The sample was prepared as described in Example 6, except that the strip of the modified ceramic sheet was 1×3 cm in size and weighed 49 mg. The treated strip weighed 399 mg, so it contained 88% of the hydrated bis(perfluorosulfonimide)oxadiazole.

The sample was 380 μm thick and 10.66 mm wide as measured for in-plane conductivity. The temperature and humidity were equilibrated at each condition before obtaining the measurements shown in Table 10. In addition, the sample was held at 120° C. and 23% relative humidity overnight for 18 hours during which it dropped in conductivity due to slow dehydration. The regain in conductivity under the final conditions indicated that the bis(perfluorosulfonimide)oxadiazole was thermally stable and could be rehydrated after prolonged exposure to high temperatures.

TABLE 10

| Time | Temperature | Relative | Resistance | Conductivity |
|---|---|---|---|---|
| hour | ° C. | Humidity % | ohm | mS/cm |
| — | 80 | 20 | 290 | 85 |
| 0 | 120 | 23 | 246 | 100 |
| 18 | 120 | 23 | 757 | 33 |
| — | 80 | 20 | 1459 | 17 |
| — | 80 | 95 | 89 | 279 |

### Example 14

A solution of 19.0 g of difluoro-fluorosulfonyl-acetyl fluoride (0.105 moles) in 250 mL toluene was prepared inside a glove box. A solution of 50 mL of 28% aqueous ammonia (0.73 moles) was diluted to 250 mL with deionized water and placed in a blender. The toluene solution was poured into the blender running at high speed. The ammonium fluoride was neutralized with 8.5 g of sodium hydroxide (0.21 moles). The aqueous phase was separated from the toluene, evaporated, and dried under vacuum. The residue was washed two times with a total of 500 mL of ethyl acetate. The extracts were dried with MgSO₄, filtered, and evaporated to give 6.75 g. The solids were redissolved in 50 mL of ethyl acetate, filtered, and evaporated to give 5.61 g (28% crude yield) of the diamide adduct, 2,2-difluoro-2-sulfamoyl-acetamide, as shown by the following structure. ¹⁹F NMR (DMSO-d₆): −110.2 (s). MS (CI): m/z 174.999111 (M+H, 1.2 ppm error), calculated 174.998895 (C₂H₄N₂O₃F₂S+H).

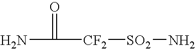

The 100 mL RBF containing 5.6 g of the diamide adduct (0.032 moles) was charged with 3.48 g of ortho-phenylene diamine (0.032 moles) and 40 mL of n-butanol. The mixture was heated to a reflux overnight under nitrogen. The white sublimate that formed in the condenser was discarded. The solvent was evaporated and the solids were dried under vacuum. The solids were recrystallized from water using decolorizing carbon. The white crystals of 2-(1H-benzoimidazol-2-yl)-2,2-difluoromethanesulfonamide, as shown by the following structure, weighed 3.05 g (38% yield). ¹H NMR (DMSO-d₆): 7.35 (m, 2H), 7.70 (m, 2H), 8.4 (bs, 2H), 13.4 (bs, 1H). ¹³C NMR (DMSO-d₆): 113.1 (C—H), 120.5 (C—H), 123.7 (2 C—H), 135.0 (C═C), 142.5 (C═C), 141.3 (t, J_{CF}=28 Hz, C—CF₂), 116.1 (t, J_{CF}=280 Hz, C—CF₂). ¹⁹F NMR (DMSO-d₆): −103.78 (s). MS (EI): m/z 247.0226 (M⁺, 0.3 ppm error), calculated 247.0227 (C₈H₇F₂N₃O₂S).

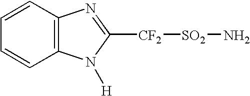

A round bottom flask equipped with a stirring bar was charged with 2.95 g of the benzimidazole perfluorosulfonamide (0.012 moles) and dissolved in 20 mL of tetrahydrofuran inside a glove box. To the flask was added 13.3 g of nonafluoro-1-butanesulfonyl fluoride (0.024 moles) and 6.7 mL of triethylamine (0.048 moles). The flask was equipped with a reflux condenser and transferred to the hood. The two-phase solution was heated to 50° C. overnight under nitrogen. The solvents were evaporated, and the resulting oil was treated with 2.5 g of sodium hydroxide (0.0625 moles) and sufficient methanol to dissolve the mixture. The solution was filtered through diatomaceous earth, which was washed with additional methanol, evaporated, and the residue was dried under vacuum to remove triethylamine. The resulting sodium salt was dissolved in a mixture of water and methanol, treated with 6 mL of concentrated hydrochloric acid, and the resulting dark oil was extracted with a 2:1 mixture of dichloromethane and methanol. The extracts were dried with magnesium sulfate, filtered, evaporated, and the resulting solids were dried under vacuum to give 5.2 g for an 82% crude yield of N-(nonafluoro-1-butanesulfonyl)-2-(1H-benzoimidazol-2-yl)-2,2-difluoromethane-sulfonamide as shown by the following structure. The benzimidazole perfluorosulfonimide was purified by dissolving in a mixture of ethyl ether and deionized water, and extracting 3 times with deionized water. The organic phase was filtered, evaporated, and dried under vacuum. The dry solids decomposed at 220° C., but melted at 72-73° C. when immersed in water. ¹H NMR (DMSO-d₆): 7.32 (m, 2H), 7.67 (m, 2H). ¹⁹F NMR (DMSO-d₆): −126.03 (m, CF₂), −121.38 (m, CF₂), −113.50 (t, J=13 Hz, CF₂), −103.37 (s, CF₂), −80.74 (t, J=9 Hz, CF₃). MS (EI): m/z 528.964825 (M⁺, 4.6 ppm error), calculated 528.962410 (C₁₂H₆F₁₁N₃O₄S₂).

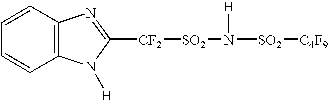

### Example 15

A glass tube was charged with 0.880 g of the benzimidazole perfluorosulfonimide from Example 14 and 0.200 g of deionized water, and analyzed for liquid conductivity. The tube was heated to 150° C. and cooled to 75° C. in 25° C. steps to obtain the measurements shown in Table 11. The sample lost 0.061 g of water. The conductivity values of this example for the benzimidazole perfluorosulfonimide are lower than those of Examples 3 and 4 for the bis(perfluorosulfonimide)oxadiazole. This supports the preference for electrolytes with an excess of acidic fluorinated sulfonyl-containing groups with respect to the aromatic heterocyclic groups, such as [1,3,4]oxadiazoles.

TABLE 11

| | Temperature, ° C. | | | |
|---|---|---|---|---|
| | 75 | 100 | 125 | 150 |
| Conductivity (heating), mS/cm | 0.33 | 0.91 | 0.66 | 0.24 |
| Conductivity (cooling), mS/cm | 0.02 | 0.10 | 0.16 | — |

### Example 16

Inside a glove box, a 300 mL round bottom flask equipped with a stirring bar, condenser, and septum was charged with 5.5 g of poly(4-vinylpyridine), 2% cross-linked (0.05 moles), and 50 mL of tetrahydrofuran. A solution of 9.3 g of difluoro-fluorosulfonyl-acetyl fluoride (0.05 moles) in 50 mL of tetrahydrofuran was prepared inside a glove box and slowly added to the round bottom flask by cannula. The mixture was cooled to 0° C. and 5.41 g of ortho-phenylene diamine was added in one portion. The mixture was then heated to a reflux for about one day. The solids were filtered off and washed with tetrahydrofuran. The filtrate was evaporated and the residue dried under vacuum. The residue was dissolved in 100 mL of water containing 4.3 g of sodium hydroxide (0.11 moles), treated with decolorizing carbon at reflux, and filtered through diatomaceous earth. The solution was concentrated and chilled overnight. A fine precipitate was filtered off and solution was acidified to pH 1 using concentrated hydrochloric acid to give a copious orange precipitate. The precipitate was filtered off and several more crops were obtained by concentrating the mother liquor. The combined solids were recrystallized from water using decolorizing carbon to give 2-(1H-benzoimidazol-2-yl) 2,2-difluoromethanesulfonic acid as shown by the following structure. Melting point: >200° C. (decomposed). ¹H NMR (DMSO-d₆): 7.48 (m, 2H), 7.77 (m, 2H), 9.1 (bs, 2H). ¹⁹F NMR (DMSO-d₆): −104.92 (s, CF₂). MS (EI): m/z 248.007316 (M⁺, 2.4 ppm error), calculated 248.006720 (C₈H₆F₂N₂O₃S).

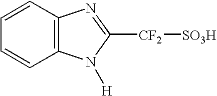

### Example 17

A pellet of 2-(1H-benzoimidazol-2-yl)-2,2-difluoromethanesulfonic acid was prepared using a hand press for the preparation of infrared spectroscopy samples. The pellet was 310 μm thick as measured for through-plane conductivity. The fixture had a short impedance of 0.166 ohms. The sample was heated to 200° C. and then cooled in 25° C. steps to obtain the measurements shown in Table 12. The results show the ability of the compound to maintain conductivity without humidification.

TABLE 12

| | Temperature, ° C. | | | | | | | |
|---|---|---|---|---|---|---|---|---|
| | 25 | 50 | 75 | 100 | 125 | 150 | 175 | 200 |
| Conductivity (heating), mS/cm | 0.64 | 0.74 | 0.76 | 1.13 | 1.38 | 3.88 | 11.3 | 16.2 |
| Conductivity (cooling), mS/cm | 22.1 | 22.3 | 23.8 | 24.8 | 25.1 | 25.6 | 25.6 | — |

### Example 18

Difluoro-fluorosulfonyl-acetyl fluoride was distilled by vacuum transfer into a Schlenk tube, weighed to give 44.0 g (0.244 moles), and dissolved in 100 mL of ether. Inside a glove box, a 1 L two-neck round bottom flask equipped with a stirring bar, gas inlet, and septum was charged with 25.6 g of poly(4-vinylpyridine), 2% cross-linked (0.244 moles), and 100 mL of ether. The solution was transferred from the Schlenk tube to the round bottom flask by cannula and cooled to 0° C. A solution of 3.90 g of hydrazine (0.122 moles) in 200 mL of ether was prepared inside a glove box and slowly added to the round bottom flask by cannula. The mixture was stirred for 1 hour, and then warmed to room temperature. The solids were filtered off and washed with ether. The filtrate was evaporated and the residue dried under vacuum. The solids were recrystallized from 200 mL of toluene using decolorizing carbon. The off-white crystals of N,N′-bis(2,2-difluoro-2-fluorosulfonyl-acetyl)hydrazine weighed 28.1 g for a 65% yield.

### Example 19

A 50 mL round bottom flask equipped with a stirring bar was charged with 12.1 g of N,N′-bis(2,2-difluoro-2-fluorosulfonyl-acetyl)hydrazine (0.035 moles) and 2.7 mL of 67.6% oleum (0.045 moles). The flask was equipped with a micro-distillation apparatus and purged with nitrogen. The apparatus was evacuated to 100 Torr and heated to 60° C. The apparatus was then evacuated to 30 Torr and heated to 120° C. A clear liquid distilled at 82-83° C. A second fraction was obtained at the same boiling point by heating the flask to 160° C. The total weight of 2,5-bis(difluoro-fluorosulfonyl-methyl)-[1,3,4]oxadiazole, as shown by the following structure, was 9.6 g for an 82% yield. The liquid fumed in air due to residual oleum. ¹⁹F NMR (CD₃CN): −99.94 (d, J=5.2 Hz, CF₂), 39.07 (t, J=5.2 Hz, SO₂F).

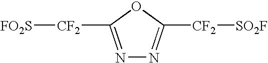

This procedure was essentially repeated using 27.75 g of N,N′-bis(2,2-difluoro-2-fluorosulfonyl-acetyl)hydrazine (0.079 moles) and 8.0 mL of 68.6% oleum (0.130 moles) to give 23.97 g for a 92% yield. The two samples were combined, washed with deionized water to remove residual oleum, and the heavy liquid drained into a 50 mL round bottom flask. Sufficient phosphorus pentoxide was added to dry the liquid, which was redistilled to give 28.48 g of 2,5-bis(difluoro-fluorosulfonyl-methyl)-[1,3,4]oxadiazole for an 85% mass balance. ¹⁹F NMR (CD₃CN): −99.94 (d, J=5.2 Hz, CF₂), 39.07 (t, J=5.2 Hz, SO₂F).

### Example 20

Difluoro-fluorosulfonyl-acetyl fluoride was distilled by vacuum transfer into a Schlenk tube and weighed to give 73.95 g (0.41 moles), and dissolved in 50 mL of ether. Inside a glove box, a 1 L two-neck round bottom flask equipped with a stirring bar, gas inlet, and septum was charged with 43.0 g of poly(4-vinylpyridine), 2% cross-linked (0.41 moles), and 150 mL of ether. The solution was transferred from the Schlenk tube to the round bottom flask by cannula and cooled to 0° C. A solution of 6.56 g of hydrazine (0.20 moles) in 300 mL of ether was prepared inside a glove box and slowly added to the round bottom flask by cannula. The mixture was stirred for 1 hour, and then warmed to room temperature. The solids were filtered off and washed with ether. The filtrate was concentrated, transferred to a 100 mL round bottom flask, evaporated, and the solids dried under vacuum to give 60.4 g of N,N′-bis(2,2-difluoro-2-fluorosulfonyl-acetyl)hydrazine for an 86% crude yield.

A stirring bar and 15 mL of 67.6% oleum (0.25 moles) was added to the flask, which was equipped with a micro-distillation apparatus. A clear liquid was vacuum distilled from the mixture at 92.5-114° C. and 37 Torr at a pot temperature of 120-150° C. The fuming liquid was washed with deionized water to remove residual oleum and the heavy liquid drained into a 100 mL round bottom flask. Sufficient phosphorus pentoxide was added to dry the liquid, which was redistilled at 92.5° C. and 37 Torr to give 42.1 g of 2,5-bis(difluoro-fluorosulfonyl-methyl)-[1,3,4]oxadiazole for an 62% pure yield. ¹⁹F NMR (CD₃CN): −99.93 (d, J=5.3 Hz, CF₂), 39.08 (t, J=5.3 Hz, SO₂F).

### Example 21

A 100 mL two-neck round bottom flask equipped with a stirring bar, septum, and dry ice condenser was purged with nitrogen, and then cooled to −78° C. Ammonia was condensed into the flask to collect about 20 mL. Inside a glove box, a 50 mL flask was charged with 3.342 g of 2,5-bis(difluoro-fluorosulfonyl-methyl)[1,3,4]oxadiazole (0.010 moles), 30 mL of acetonitrile, and sealed with a septum. The solution was added to the ammonia by cannula under nitrogen. The solution was stirred for 1 hour, warmed to room temperature, and stirred overnight under nitrogen. The mixture was treated with 30 mL of acetonitrile, 30 mL of 2M hydrogen chloride in ether, and 1.2 g of calcium chloride (0.011 moles). The solids that precipitated were filtered off and washed with acetonitrile and ether. The solvents were evaporated and the residue was dried under vacuum to give 3.00 g for a 92% crude yield. The mixture was subjected to chromatography on silica gel using 20% acetone in dichloromethane and the common fractions were evaporated to give 0.4 g (12% yield) of 2,5-bis(difluoro-sulfamoyl-methyl)-[1,3,4]oxadiazole as shown by the following structure. Several samples of similar purity were combined and repurified by chromatography to give 1.12 g of pure material. Melting point: 171-172° C. ¹⁹F NMR (CD₃CN): −106.64 (s, CF₂). MS (CI): m/z 328.963722(M+H⁺, 0.1 ppm error), calculated 328.963751 (C₄H₄N₄O₅F₄S₂+H⁺).

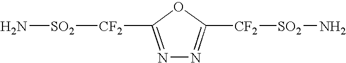

### Example 22

Inside a glove box, a 50 mL round bottom flask equipped with a stirring bar, condenser, and septum was charged with 1.066 g of octafluoro-1,4-butanedisulfonyl difluoride (0.00291 moles), 0.956 g of 2,5-bis(difluoro-sulfamoyl-methyl)-[1,3,4]oxadiazole (0.00291 moles), and 10 mL of acetonitrile. To the flask was added 2.1 mL of triethylamine (0.015 moles). The solution was stirred for 1 hour, then heated to a reflux overnight under nitrogen. The mixture was treated with 0.7 mL of 50% sodium hydroxide. The solvents were evaporated and the residue dried under vacuum to give 1.75 g. The residue was dissolved in deionized water, treated with decolorizing carbon, and filtered through diatomaceous earth, which was washed with additional water. The aqueous solution was subjected to ion exchange following the general procedure in Example 2 to give a viscous oil. The oil was dissolved in deionized water and filtered with washing through a 0.45 μm PTFE membrane to remove some fine dark solids. The water was evaporated to give 1.03 g of lightly colored oil. The oil was dissolved in 100 mL of deionized water and subjected to ion exchange following the general procedure of Example 2, except that the column contained 20 g of Dowex® resin. The final lightly colored hydrated oil of the desired poly(oxadiazole perfluorosulfonimide), as shown by the following structure, weighed 1.33 g. MALDI mass spectroscopy showed molecular weights of up to 2700 daltons.

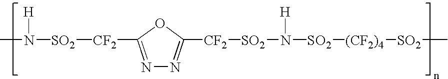

### Example 23

A 1×3 cm strip of the modified ceramic fiber substrate from Example 6 was cut from the stock sheet and weighed 48 mg. The hydrated oil of the poly(oxadiazole perfluorosulfonimide) from Example 22 was diluted with deionized water until fluid. The strip was laid inside a vial and immersed in the oil, then dried in a cool vacuum oven under nitrogen purge. The treated strip weighed 246 mg, so it contained 80% of the hydrated polymer.

The sample was 170 μm thick as measured for through-plane conductivity. The fixture had a short impedance of 0.280 ohms. The sample was heated to 150° C. and then cooled in 25° C. steps to obtain the measurements shown in Table 13. The results show the ability of the polymer to maintain conductivity without humidification and to retain water at elevated temperatures.

TABLE 13

| | Temperature, ° C. | | | | | |
|---|---|---|---|---|---|---|
| | 25 | 50 | 75 | 100 | 125 | 150 |
| Conductivity (heating), mS/cm | 4.6 | 6.5 | 12 | 20 | 29 | 36 |
| Conductivity (cooling), mS/cm | 3.6 | 12 | 19 | 25 | 32 | — |

### Example 24

The remaining treated strip from Example 23 was used to prepare another sample for measurement of through-plane conductivity, except that a drop of deionized water was added to the sample before compressing the stack.

The sample was 170 μm thick as measured for through-plane conductivity. The fixture had a short impedance of 0.280 ohms. The sample was heated to 150° C. and then cooled in 25° C. steps to obtain the measurements shown in Table 14. The higher conductivities seen on the heating cycle as compared to Example 23 shows the effect of partial hydration of the polymer and its ability to retain water at elevated temperatures.

TABLE 14

| | Temperature, ° C. | | | | | |
|---|---|---|---|---|---|---|
| | 25 | 50 | 75 | 100 | 125 | 150 |
| Conductivity (heating), | 93 | 108 | 143 | 169 | 151 | 68 |
| mS/cm | | | | | | |
| Conductivity (cooling), | 4.8 | 11 | 16 | 21 | 23 | — |
| mS/cm | | | | | | |

### Example 25

A 500 mL two-neck round bottom flask equipped with a stirring bar, septum, and jacketed addition funnel with dry ice condenser was charged with 60 mL of acetonitrile, purged with nitrogen, and cooled to −40° C. Ammonia was condensed into the funnel to collect 4.5 mL (0.18 moles) and added to the flask. Inside a glove box, a 125 mL flask was charged with 10.02 g of 2,5-bis(difluoro-fluorosulfonyl-methyl)-[1,3,4]oxadiazole (0.030 moles), 90 mL of acetonitrile, and sealed with a septum. The solution was added dropwise by cannula to the ammonia at −40° C. to give a precipitate. The mixture was stirred for 1 hour, warmed to room temperature, and stirred for 1 hour. The mixture was treated with 30 mL of 2M hydrogen chloride in ether and 1.2 g of calcium chloride. The resulting solids were filtered off and washed twice with acetonitrile. The solution was evaporated and the residue was dried under vacuum. The residue weighed 9.83 g for a 100% crude yield. The mixture was subjected to chromatography on silica gel using 60:40 ethyl acetatehexane to give 5.16 g of 2,5-bis(difluoro-sulfamoyl-methyl)-[1,3,4]oxadiazole for a 52% pure yield. ¹⁹F NMR (CD₃CN): −106.62 (s, CF₂). Several samples of similar purity were combined for a total of 7.5 g and recrystallized from anisole using decolorizing carbon to give 6.88 g for a 92% mass balance.

### Example 26

Inside a glove box, a 100 mL round bottom flask equipped with a stirring bar, condenser, and septum was charged with 2.297 g of 2,& bis(difluoro-sulfamoyl-methyl)-[1,3,4]oxadiazole (0.007 moles), 2.563 g of octafluoro-1,4-butanedisulfonyl difluoride (0.007 moles), and 20 mL of acetonitrile. To the flask was added 4.2 mL of triethylamine (0.03 moles). The solution was stirred for 4 hours then heated to a reflux under nitrogen. After two nights, the mixture was cooled to room temperature, treated with 1.2 g of sodium hydroxide (0.03 moles) and 3.4 g of calcium chloride (0.03 moles), and heated briefly to a reflux. The solids were filtered off and washed with acetonitrile. The solution was evaporated and the residue dried under vacuum. The residue was dissolved in 100 mL of deionized water at reflux by adding sufficient sodium hydroxide to dissolve any remaining solids. The hot solution was treated with decolorizing carbon, diatomaceous earth, and then filtered through a 5 μm PTFE membrane filter. After evaporation, the residue contained triethylamine according to ¹H NMR (CD₃CN). The solids were dissolved in 500 mL of deionized water and the aqueous solution was subjected to ion exchange following the general procedure in Example 2 to give hygroscopic solids that were free of triethylamine. The ion-exchange procedure was repeated to insure complete proton exchange and the resulting solids were dried under vacuum to give 4.36 g for a 95% yield of the desired poly(oxadiazole perfluorosulfonimide). ¹⁹F NMR (CD₃CN): −120.96 (m, CF₂), −113.73 (m, CF₂), −110.56 (s, CF₂).

### Example 27

A 1×3 cm strip was cut from glass microfiber filter paper (Acid Treated Low Metal TCLP filters, Whatman International Ltd., Maidstone, England) and weighed 24 mg. The poly(oxadiazole perfluorosulfonimide) from Example 26 was added to a vial and dissolved in minimal deionized water. The strip was laid inside the vial and immersed in the solution, then dried in a cool vacuum oven under nitrogen purge. The treated strip weighed 259 mg, so it contained 91% of the hydrated polymer.

The sample was 350 μm thick and 10.13 mm wide as measured for in-plane conductivity. The temperature and humidity were equilibrated at each condition before obtaining the measurements shown in Table 15.

The results show the ability of the poly(oxadiazole perfluorosulfonimide) to maintain high conductivity even at low relative humidity.

TABLE 15

| Temperature | Relative | Pressure | Resistance | Conductivity |
|---|---|---|---|---|
| ° C. | Humidity % | psia | ohm | mS/cm |
| 120 | 10 | 14.7 | 546 | 52 |
| 120 | 25 | 35.0 | 181 | 156 |
| 120 | 50 | 35.2 | 83 | 340 |
| 120 | 95 | 35.5 | 40 | 711 |

### Example 28

A 1×1 cm strip was cut from glass microfiber filter paper (Acid Treated Low Metal TCLP filters, Whatman International Ltd., Maidstone, England) and weighed 8 mg. The poly(oxadiazole perfluorosulfonimide) from Example 26 was added to a vial and dissolved in minimal deionized water. The strip was laid inside the vial and immersed in the solution, then dried in cool vacuum oven under nitrogen purge. The treated strip weighed 123 mg, so it contained 93% of the hydrated polymer.

The sample was 150 μm thick as measured for through-plane conductivity. The fixture had a short impedance of 0.341 ohms. The sample was heated to 150° C. in 25° C. steps to obtain the measurements shown in Table 16. The results show the ability of the poly(oxadiazole perfluorosulfonimide) to retain water at elevated temperatures and maintain conductivity without humidification.

TABLE 16

| | Temperature, ° C. | | | | | |
|---|---|---|---|---|---|---|
| | 25 | 50 | 75 | 100 | 125 | 150 |
| Conductivity, mS/cm | 27 | 36 | 61 | 98 | 142 | 189 |

1. A compound having the general structure: 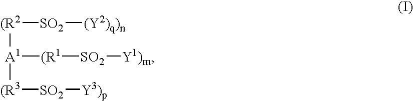
wherein A¹ is a monovalent, divalent, or trivalent aromatic heterocyclic group comprising heterocyclic rings
R¹, R², and R³ are divalent fluorinated groups;
m, n, and p are 0 to 3, with the proviso that m+n+p is equal to 1, 2, or 3 so that the carbon atoms of the heterocyclic rings are fully substituted by acidic fluorinated sulfonyl-containing groups;
q is 0 or 1;
Y¹ is —OH, —NH—SO₂—R⁴ wherein R⁴ is a monovalent fluorinated group, —NH—, —NH—SO₂—R⁵—SO₂—NH—, or —NH—SO₂—R⁶-A²-R⁷—SO₂—NH—, wherein A² is a divalent heterocyclic group and R⁵, R⁶, and R⁷ are divalent fluorinated groups; and
Y² and Y³ are —OH or —NH—SO₂—R⁴; with the proviso that when m and n are each equal to 1, p is 0 to 1, and q is 0, Y¹ is selected from the group consisting of —NH—, —NH—SO₂—R⁵—SO₂—NH—, and —NH—SO₂—R⁶-A²-R⁷—SO₂—NH—.

2. The compound of claim 1 wherein the compound is a small molecule.

3. The compound of claim 1 wherein the compound is a repeat unit for a polymer.

4. The compound of claim 1, 2 or 3 wherein A¹ selected from the group consisting of oxadiazole, triazole, thiadiazole, pyrazole, triazine, tetrazole, oxazole, thiazole, imidazole, benzoxazole, benzothiazole, benzimidazole, benzobisoxazole, benzobisthiazole, benzobisimidazole, bibenzoxazole, bibenzothiazole, and bibenzimidazole.

5. The compound of claim 3 wherein A¹ is selected from the group consisting of [1,3,4]oxadiazole, [1,3,4]thiadiazole, and [1,2,4]triazole.

6. The compound of claim 5 wherein A¹ is [1,3,4]oxadiazole.

7. The compound of claim 3 wherein R¹, R², and R³ are linear, branched, or cyclic perfluorinated or partially fluorinated saturated or unsaturated groups having 1 to 20 carbon atoms optionally containing ethereal oxygen, chlorine, bromine, or iodine atoms.

8. The compound of claim 7 wherein R¹, R², and R³ are linear or branched perfluorinated saturated or unsaturated groups having 1 to 10 carbon atoms optionally containing ethereal oxygen atoms.

9. The compound of claim 8 wherein R¹, R², and R³ are linear perfluorinated saturated groups having 1 to 6 carbon atoms.

10. The compound of claim 1, 2, or 3 wherein m+n+p is equal to 2 or 3.

11. The compound of claim 3 wherein m+n+p is equal to 2.

12. The compound of claim 1 or 3 wherein A² is a divalent aromatic heterocyclic group, such as an oxadiazole, triazole, thiadiazole, benzobisoxazole, benzobisthiazole, benzobisimidazole, bibenzoxazole, bibenzothiazole, and bibenzimidazole.

13. The compound of claim 3 wherein A² is [1,3,4]oxadiazole.

14. The compound of claim 1 or 3 wherein R⁵, R⁶, and R⁷ are linear, branched, or cyclic perfluorinated or partially fluorinated saturated or unsaturated groups having 1 to 20 carbon atoms optionally containing ethereal oxygen, chlorine, bromine, or iodine atoms.

15. The compound of claim 1 or 2 wherein Y¹, Y², and Y³ are each equal to —OH or —NH—SO₂—R⁴, wherein R⁴ is any monovalent fluorinated group, and q is 1.

16. The compound of claim 1 wherein R⁴ is a linear, branched, or cyclic perfluorinated or partially fluorinated saturated or unsaturated group having 1 to 20 carbon atoms optionally containing ethereal oxygen, chlorine, bromine, or iodine atoms.

17. The compound of claim 1 wherein m+n+p is equal to 2 or 3.

18. The compound of claim 1 or 2 wherein Y¹ is —NH—SO₂—R⁴, n and p are each equal to 0, and m is 2 or 3.

19. The compound of claim 3 wherein m and n is each equal to 1, p is 0 to 1, and q is 0.

20. The compound of claim 19 wherein A¹ is a divalent aromatic heterocyclic group, m and n are each equal to 1, p is 0, q is 0, and Y¹ is —NH—.

21. The compound of claim 19 wherein A¹ is a divalent aromatic heterocyclic group, m and n are each equal to 1, p is 0, q is 0, and Y¹ is —NH—SO₂—R⁵—SO₂—NH—, wherein R⁵ is a divalent fluorinated group.

22. The compound of claim 19 wherein A¹ is a divalent aromatic heterocyclic group, m and n are each equal to 1, p is 0, q is 0, and Y¹ is —NH—SO₂—R⁶-A²-R⁷—SO₂—NH—, wherein R⁶ and R⁷ are a divalent fluorinated groups.

23. A compound of claim 3 wherein the compound is a random copolymer obtained by randomly combining any variety of the polymer repeat units, in any ratio with respect to each other, wherein m and n are each equal to 1, p is 0 to 1 and q is 0.

24. A compound of claim 1 or 2 wherein A¹ is a divalent aromatic heterocyclic group, m is 2, n and p are each equal to 0, and Y¹ is —NH—SO₂—R⁴.

25. A compound of claim 1 or 3 wherein A¹ is a divalent aromatic heterocyclic group, m and n are each equal to 1, p is 0, q is 0, and Y¹ is —NH—.

26. A compound of claim 1 or 3 wherein A¹ is a divalent aromatic heterocyclic group, m and n are each equal to 1, p is 0, q is 0, and Y¹ is —NH—SO₂—R⁵—SO₂—NH—.

27. A compound of claim 1 or 3 wherein A¹ is a divalent aromatic heterocyclic group, m and n are each equal to 1, p is 0, q is 0, and Y¹ is —NH—SO₂—R⁶-A²-R⁷—SO₂—NH—.

28. A fluorinated fluorosulfonyl-substituted heterocycle having the general structure: 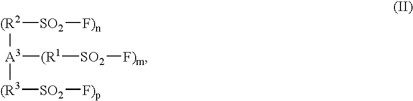
wherein A³ is a divalent or trivalent aromatic heterocyclic group comprising heterocyclic rings;
R¹, R², and R³ are divalent fluorinated groups;
m, n, and p are 0 to 3, with the proviso that m+n+p is equal to 2 or 3 so that the carbon atoms of the heterocyclic rings are fully substituted by fluorinated fluorosulfonyl groups.

29. The fluorinated fluorosulfonyl-substituted heterocycle of claim 28 wherein A³ is a divalent aromatic heterocyclic group, m and n are each equal to 1, and p is 0.

30. The fluorinated fluorosulfonyl-substituted heterocycle of claim 28 wherein A³ is a divalent aromatic heterocyclic group, n and p are each equal to 0, and m is 2.

31. A process for synthesizing a compound comprising the following steps:
(a) providing a fluorosulfonyl-containing acyl derivative having the structure:
F—SO₂—R⁸—X,
wherein R⁸ is a divalent fluorinated group as defined above for R¹ and X is an acyl group;
(b) condensing the fluorosulfonyl-containing acyl derivative from step (a) with a nitrogenous reagent to form a sulfonyl-containing precursor;
(c) cyclizing the sulfonyl-containing precursor of step (b) by thermolysis or dehydration to form a sulfonyl-containing aromatic heterocyclic compound containing fluorosulfonyl groups or sulfonamide groups; and
(d) converting the sulfonyl-containing aromatic heterocyclic compound of step (c) containing fluorosulfonyl groups or sulfonamide groups, into an acidic sulfonyl-containing aromatic heterocyclic compound by either:
(i) condensing fluorosulfonyl groups with a fluorinated sulfonamide,
(ii) condensing sulfonamide groups with a fluorinated sulfonyl fluoride,
(iii) condensing fluorosulfonyl groups first with ammonia to form sulfonamide groups followed by a fluorinated sulfonyl fluoride to form sulfonimide groups, or
(iv) hydrolysis of fluorosulfonyl or sulfonamide groups to form sulfonic acid groups.

32. The process of claim 31 wherein the acyl group is selected from the group consisting of acyl fluoride, acyl chloride, acyl bromide, acyl iodide, an ester, an amide, and nitrile.

33. The process of claim 31 wherein the nitrogenous reagent, is selected from the group consisting of ammonia; hydrazine; an azide; and an organic ortho-substituted aromatic amine.

34. A process for synthesizing a bis(sulfonimide)-[1,3,4]oxadiazole by condensing a fluorosulfonyl acyl fluoride, F—SO₂—R⁸—CO—F, with hydrazine to form a bis(fluorosulfonyl)dihydrazide containing a dihydrazide group and fluorosulfonyl groups; forming a [1,3,4]oxadiazole ring by cyclizing the dihydrazide group using dehydration; condensing the fluorosulfonyl groups with ammonia to form a bis(sulfonamide)-[1,3,4]oxadiazole containing sulfonamide groups; and forming sulfonimide groups by condensing a fluorinated sulfonyl fluoride, R⁴—SO₂—F, with the sulfonamide groups, wherein R⁴ and R⁸ are linear perfluorinated saturated groups having 1 to 6 carbon atoms.

35. A process for synthesizing a copolymer containing sulfonimide and [1,3,4]oxadiazole groups by condensing a fluorosulfonyl acyl fluoride, F—SO₂—R⁸—CO—F, with hydrazine to form a bis(fluorosulfonyl)dihydrazide containing a dihydrazide group and fluorosulfonyl groups; forming a [1,3,4]oxadiazole ring by cyclizing the dihydrazide group using dehydration; condensing the fluorosulfonyl groups with ammonia to form a bis(sulfonamide)-[1,3,4]oxadiazole containing sulfonamide groups; and forming sulfonimide groups by condensing a fluorinated disulfonyl difluoride, F—SO₂—R⁵—SO₂—F, with the sulfonamide groups, wherein R⁵ and R⁸ are linear perfluorinated saturated groups having 1 to 6 carbon atoms.

36. A process for synthesizing a benzimidazole sulfonimide by condensing a fluorosulfonyl acyl fluoride, F—SO₂—R⁸—CO—F, with ammonia to form a diamide containing a carbamide group and a sulfonamide group; condensing the carbamide group with an ortho-phenylene diamine to form a carbamide adduct; cyclizing the carbamide adduct by thermolysis to form a benzimidazole group, and forming a sulfonimide group by condensing a fluorinated sulfonyl fluoride, R⁴—SO₂—F, with the sulfonamide group, wherein R⁴ and R⁸ are linear perfluorinated saturated groups having 1 to 6 carbon atoms.

37. A process for synthesizing a benzimidazole sulfonic acid by condensing a fluorosulfonyl acyl fluoride, F—SO₂—R⁸—CO—F, with an ortho-phenylene diamine to form a carbamide adduct; cyclizing the carbamide adduct by thermolysis to form a benzimidazole group, and forming a sulfonic acid group by hydrolyzing the fluorosulfonyl group wherein R⁸ is a linear perfluorinated saturated group having 1 to 6 carbon atoms.

38. A solid polymer electrolyte membrane comprising a porous substrate having imbibed therein a compound having the general structure: 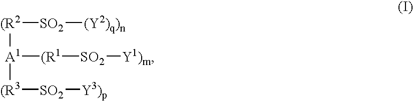
wherein A¹ is a monovalent, divalent, or trivalent aromatic heterocyclic group comprising heterocyclic rings;
R¹, R², and R³ are divalent fluorinated groups;
m, n, and p are 0 to 3, with the proviso that m+n+p is equal to 1, 2, or 3 so that the carbon atoms of the heterocyclic rings are fully substituted by acidic fluorinated sulfonyl-containing groups;
q is 0 or 1;
Y¹ is —OH, —NH—SO₂—R⁴ wherein R⁴ is a monovalent fluorinated group, —NH—, —NH—SO₂—R⁵—SO₂—NH—, or —NH—SO₂—R⁶-A²-R⁷—SO₂—NH—, wherein A² is a divalent aromatic heterocyclic group and R⁵, R⁶, and R⁷ are divalent fluorinated groups; and
Y² and Y³ are —OH or —NH—SO₂—R⁴; with the proviso that when m and n are each equal to 1, p is 0 to 1, and q is 0, Y¹ is selected from the group consisting of —NH—, —NH—SO₂—R⁵—SO₂—NH—, and —NH—SO₂—R⁶-A²-R⁷—SO₂—NH—.

39. The solid polymer electrolyte membrane of claim 38 wherein the porous substrate is selected from the group consisting of inorganic fiber substrates and microporous films of perfluorinated polymers.

40. The solid polymer electrolyte membrane of claim 38 wherein the compound is a small molecule.

41. The solid polymer electrolyte membrane of claim 38 wherein the compound is a repeat unit for a polymer.

42. The solid polymer electrolyte membrane of claim 38 wherein the compound is cross linked, grafted, or chain extended within the porous support.

43. The solid polymer electrolyte membrane of claim 42 wherein the compound is modified to contain reactive functional groups to provide crosslinking, grafting, or chain extension.

44. The solid polymer electrolyte membrane of claim 42 wherein the compound is mixed with reagents to provide crosslinking, grafting, or chain extension.

45. A catalyst coated membrane comprising a solid polymer electrolyte membrane having a first surface and a second surface, an anode present on the first surface of the solid polymer electrolyte membrane, and a cathode present on the second surface of the solid polymer electrolyte membrane, wherein the solid polymer electrolyte membrane comprises a porous substrate having imbibed therein a compound having the general structure: 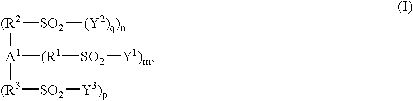
wherein A¹ is a monovalent, divalent, or trivalent aromatic heterocyclic group comprising heterocyclic rings;
R¹, R², and R³ are divalent fluorinated groups;
m, n, and p are 0 to 3, with the proviso that m+n+p is equal to 1, 2, or 3 so that the carbon atoms of the heterocyclic rings are fully substituted by acidic fluorinated sulfonyl-containing groups;
q is 0 or 1;
Y¹ is —OH, —NH—SO₂—R⁴ wherein R⁴ is a monovalent fluorinated group, —NH—, —NH—SO₂—R⁵—SO₂—NH—, or —NH—SO₂—R⁶-A²-R⁷—SO₂—NH—, wherein A² is a divalent aromatic heterocyclic group and R⁵, R⁶, and R⁷ are divalent fluorinated groups; and
Y² and Y³ are —OH or —NH—SO₂—R⁴; with the proviso that when m and n are each equal to 1, p is 0 to 1, and q is 0, Y¹ is selected from the group consisting of —NH—, —NH—SO₂—R⁵—SO₂—NH—, and —NH—SO₂—R⁶-A²-R⁷—SO₂—NH—.

46. A membrane electrode assembly comprising a polymer electrolyte membrane having a first surface and a second surface, and comprising a compound having the general structure: 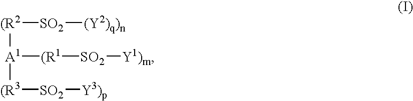
wherein A¹ is a monovalent, divalent, or trivalent aromatic heterocyclic group comprising heterocyclic rings;
R¹, R², and R³ are divalent fluorinated groups;
m, n, and p are 0 to 3, with the proviso that m+n+p is equal to 1, 2, or 3 so that the carbon atoms of the heterocyclic rings are fully substituted by acidic fluorinated sulfonyl-containing groups;
q is 0 or 1;
Y¹ is —OH, —NH—SO₂—R⁴ wherein R⁴ is a monovalent fluorinated group, —NH—, —NH—SO₂—R⁵—SO₂—NH—, or —NH—SO₂—R⁶-A²-R⁷—SO₂—NH—, wherein A² is a divalent aromatic heterocyclic group and R⁵, R⁶, and R⁷ are divalent fluorinated groups; and
Y² and Y³ are —OH or —NH—SO₂—R⁴; with the proviso that when m and n are each equal to 1, p is 0 to 1, and q is 0, Y¹ is selected from the group consisting of —NH—, —NH—SO₂—R⁵—SO₂—NH—, and —NH—SO₂—R⁶-A²-R⁷—SO₂—NH—.

47. An electrocatalyst coating composition comprising a compound having the general structure: 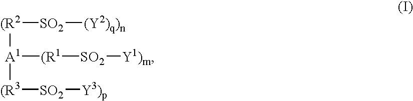
wherein A¹ is a monovalent, divalent, or trivalent aromatic heterocyclic group comprising heterocyclic rings;
R¹, R², and R³ are divalent fluorinated groups;
m, n, and p are 0 to 3, with the proviso that m+n+p is equal to 1, 2, or 3 so that the carbon atoms of the heterocyclic rings are fully substituted by acidic fluorinated sulfonyl-containing groups;
q is 0 or 1;
Y¹ is —OH, —NH—SO₂—R⁴ wherein R⁴ is a monovalent fluorinated group, —NH—, —NH—SO₂—R⁵—SO₂—NH—, or —NH—SO₂—R⁶-A²-R⁷—SO₂—NH—, wherein A² is a divalent aromatic heterocyclic group and R⁵, R⁶, and R⁷ are divalent fluorinated groups; and
Y² and Y³ are —OH or —NH—SO₂—R⁴; with the proviso that when m and n are each equal to 1, p is 0 to 1, and q is 0, Y¹ is selected from the group consisting of —NH—, —NH—SO₂—R⁵—SO₂—NH—, and —NH—SO₂—R⁶-A²-R⁷—SO₂—NH—.

48. An electrocatalyst coating composition of claim 47 further comprising a catalyst.

49. An electrochemical cell comprising a polymer electrolyte membrane, wherein the polymer electrolyte membrane comprises a compound having the general structure: 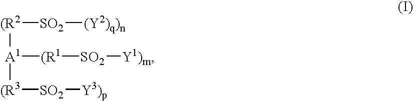
wherein A¹ is a monovalent, divalent, or trivalent aromatic heterocyclic group comprising heterocyclic rings;
R¹, R², and R³ are divalent fluorinated groups;
m, n, and p are 0 to 3, with the proviso that m+n+p is equal to 1, 2, or 3 so that the carbon atoms of the heterocyclic rings are fully substituted by acidic fluorinated sulfonyl-containing groups;
q is 0 or 1;
Y¹ is —OH, —NH—SO₂—R⁴ wherein R⁴ is a monovalent fluorinated group, —NH—, —NH—SO₂—R⁵—SO₂—NH—, or —NH—SO₂—R⁶-A²-R⁷—SO₂—NH—, wherein A² is a divalent aromatic heterocyclic group and R⁵, R⁶, and R⁷ are divalent fluorinated groups; and
Y² and Y³ are —OH or —NH—SO₂—R⁴; with the proviso that when m and n are each equal to 1, p is 0 to 1, and q is 0, Y¹ is selected from the group consisting of —NH—, —NH—SO₂—R⁵—SO₂—NH—, and —NH—SO₂—R⁶-A²-R⁷—SO₂—NH—.

50. The electrochemical cell of claim 49 selected from the group consisting of fuel cells, batteries, chloralkali cells, electrolysis cells, sensors, electrochemical capacitors, and modified electrodes.
